(19) United States
(12) Patent Application Publication
(10) Pub. No.: US 2021/0340595 A1
(43) Pub. Date: Nov. 4, 2021

(54) Blood Sample Processing and Nucleic Acid Amplification Systems, Devices, and Methods

(71) Applicant: The Trustees of Columbia University in the City of New York, New York, NY (US)

(72) Inventors: Samuel K. SIA, New York, NY (US); Nicole R. BLUMENFELD, New York, NY (US); Rodrigo C. CHAVES, New York, NY (US); Giuseppe MODICA, New York, NY (US); Michael LUETOLF, New York, NY (US)

(73) Assignee: The Trustees of Columbia University in the City of New York, New York, NY (US)

(21) Appl. No.: 17/210,083

(22) Filed: Mar. 23, 2021

Related U.S. Application Data
(63) Continuation of application No. PCT/US2019/052844, filed on Sep. 25, 2019.
(60) Provisional application No. 62/736,033, filed on Sep. 25, 2018.

(51) Int. Cl. C12Q 1/6806; C12N 15/10

(52) U.S. Cl. CPC C12Q 1/6806; C12N 15/1017

(57) ABSTRACT

Aspects described herein provide blood sample purification and microfluidic nucleic acid amplification systems and methods for purifying blood samples and amplifying a target nucleic acid on the same system. The described systems and methods include a filter component and a microfluidic amplification component for amplification of nucleic acid from the blood sample, and can be used, for example, for point of care blood analysis.

### CROSS-REFERENCE TO RELATED APPLICATIONS

This application is a continuation of International Application No. PCT/US2019/052844 filed on Sep. 25, 2019, which claims priority to and the benefit of U.S. Provisional Patent Application No. 62/736,033 filed Sep. 25, 2018, both of which are hereby incorporated by reference in their entirety.

### BACKGROUND

Current sample preparation methods for processing whole blood involve large, laboratory-based pieces of equipment, high cost, and trained users. For example, a standard laboratory centrifuge, which is required for plasma extraction from whole blood, can cost upwards of $4,000 and take up to several square feet of space and must be operated by a trained user. In addition, filtering large volumes of blood (ml and above) clogs filtering devices, making filtering an impractical alternative to use of centrifugation.

Similarly, nucleic acid purification typically occurs via several pieces of laboratory equipment, including but not limited to, vacuums, specialized tubing, specialized buffers, centrifuges and vortexes.

Currently available filtering devices, that do not have the advantages of aspects described herein include, PlasmaDrop PD-50 (MDI Membrane), Blood Separator (Nupore), Vivid Plasma Separation Membrane (Pall). See, also, Wang, S. et al. (2012). Simple filter microchip for rapid separation of plasma and viruses from whole blood, Int J Nanomedicine. 7: 5019-5028. DOI: 10.2147/IJN.532579.

### SUMMARY

Objects and advantages of embodiments of the disclosed subject matter will become apparent from the following description when considered in conjunction with the accompanying drawings.

Aspects described herein combine portable sample preparation (e.g. from whole blood) and downstream processing (e.g., nucleic acid amplification) from the purified sample. Further aspects provide an optionally low-cost, portable, plasma extraction device that is capable of utilizing minimal power consumption. Aspects described herein integrate, for example, sample preparation and nucleic amplification into a plastic microfluidic chip. Use of these aspects can be automated, is low cost, uses minimal volumes of fluid (e.g., whole blood) and does not require a trained user to carry out each step. Combining these two components (plasma extraction and nucleic acid purification) is further beneficial, as these two steps are typically performed sequentially in a lab and must be performed prior to almost all laboratory nucleic acid assays.

As described herein, the exemplary 2-membrane design, selectively filters out cells and enables processing of large volumes of blood (e.g., about 100 milliliters and above) without clogging the whole filter. Conventionally, microfluidic analysis with large volumes of sample is extremely difficult because simple filters will get clogged. Conventionally, filters are available on market are recommended to be used individually, not together, because it was thought that conventional use would increase chances of clogging as well as increased sample loss. In addition, it was thought that stacking filters on top of each other would result in folding and sticking of the filters to each other, resulting in decreased or entire loss of filtration function and putting a large space between two filters would increase the dead volume and efficiency of the process.

### BRIEF DESCRIPTION OF DRAWINGS

FIG. 1 shows an exemplary multi-stage filter system and flowchart for separation of plasma components;

FIG. 2A shows an exemplary middle section filter support design with a top and bottom view (A.1), side view (A.2), and 3D view (A.3);

FIG. 2B shows an exemplary top and bottom filter support design with a top view (B.1) bottom view (B.2), side view (B.3), 3D front-side view (B.4), and 3D back-side view (B.5);

FIG. 3A shows an exemplary filter support design with a top section having an outlet, a middle section and bottom section having an inlet and two exemplary filters having different pore sizes to be disposed between the top and middle section and bottom and middle section;

FIG. 3B provides a photograph of an exemplary assembled device;

FIG. 4 provides a photograph of an exemplary assembled prototype (prototype #3) with a syringe mounted in a syringe pump;

FIG. 5 provides a 3D view of an exemplary modified design for the bottom and top part containing a groove;

FIG. 6 shows an alternate 3D view of the exemplary modified design shown in FIG. 5 with top, middle, and bottom parts and exemplary filter membranes;

FIG. 7A shows an exemplary plasma separation apparatus with a double stage filter, collection tube, and pump;

FIG. 7B shows plasma collected from whole blood (B.1), plasma collected from 1:4 diluted sample (B.2), plasma collected from 1:6 diluted sample (B.3), and plasma collected from 1:8 diluted sample (B.4);

FIG. 8 provides the results of an exemplary separation efficiency study of the collected plasma samples from the blood samples of FIG. 7B;

FIG. 9 provides the results of an exemplary test for hemoglobin presence from the blood samples of FIG. 7B showing absorbance values at wavelengths of 540 nm and 576 nm;

FIG. 10 provides photographs of an initial blood sample and filtered collected samples at different flow: (A.1) 1:6 diluted blood input sample, (A.2) plasma collected at 100 μl/min, (A.3) Plasma collected at 150 μl/min, (A.4) Plasma collected at 200 μl/min;

FIG. 11 provides the separation efficiency of the collected plasma samples at a 1:6 diluted input blood sample at the indicated flow rate;

FIG. 12 provides the results of an exemplary test for hemoglobin presence from the blood samples of FIG. 10 at 540 nm and at 576 nm;

FIG. 13 provides photographs of an input sample and collected outputs with different combination of membranes—(A.1) 1:6 diluted blood input sample, (A.2) plasma collected by “5 μm+3 μm” combination, (A.3) plasma collected by “5 μm+2 μm” combination, and (A.4) Plasma collected by “3 μm+2 μm” combination;

FIG. 14 provides the results of an exemplary separation efficiency study of the collected plasma samples from the blood samples of FIG. 13 for different combination of membranes, as indicated;

FIG. 15 provides the results of an exemplary test for hemoglobin presence from the blood samples of FIG. 13 at 540 nm and at 576 nm at 540 nm and at 576 nm for the collected plasma samples for different combination of membranes, as indicated;

FIG. 16 shows the results of comparative standard and filtered PCR products run in a 2% agarose gel;

FIG. 17 shows the results of comparative standard and filtered PCR products run in a 2% agarose gel;

FIG. 18 provides an exemplary schematic view of the smaller version for the double stage filter plasma separation device;

FIG. 19 is a photograph of an exemplary plasma separation device;

FIG. 20 shows the results of comparative standard and filtered PCR products run in a 2% agarose gel;

FIG. 21 shows the results of testing voltage (DC) versus flow rate for a peristaltic pump;

FIG. 22 shows comparative separation efficiency different device configurations compared to a centrifuge control;

FIG. 23 shows the degree of hemolysis as measured at 540 nm (left) and 576 nm (right;)

FIG. 24 shown exemplary PCR amplification curves from plasma samples at various concentrations;

FIG. 25 shows an exemplary PMMA microfluidic device for DNA amplification;

FIG. 26 provides additional views of the exemplary PMMA microfluidic device for DNA amplification;

FIG. 27 shows an exemplary 3D printed valve holder;

FIG. 28 shows how solutions can be mixed inside the exemplary microfluidic device;

FIG. 29 shows an absorption spectrum of DNA extraction on the exemplary microfluidic device;

FIG. 30 shows nucleic acid concentration distribution for an exemplary nucleic acid extraction;

FIG. 31A shows comparative extraction efficiency comparison between silica magnetic beads tube, silica column and silica magnetic beads device and relative standard deviation;

FIG. 31B shows comparative nucleic acid extracted concentration comparison between silica magnetic beads tube, silica column and silica magnetic beads device;

FIG. 32 shows extraction efficiency vs. time progression;

FIG. 33 shows exemplary gel electrophoresis results from DNA solid phase extraction (SPE) samples after PCR;

FIG. 34 provides an exemplary Solidworks® design of a microfluidic chip with three different inlets, an extraction chamber, a PCR chamber. and a waste channel;

FIG. 35A shows an exemplary holder for valves, and FIG. 35B shows an electronic circuit for multiple valve actuation;

FIG. 36A illustrates the working principle of the valve in an open state, and FIG. 36B illustrates the working principle of the valve in a closed state;

FIG. 37A provides an exemplary 3D drawing of the exemplary valve, and FIG. 37B provides a photograph of the valve taken under the stereoscope;

FIG. 38A shows the deflection of the PDMS membrane as a function of the radius for different membrane thicknesses, and FIG. 38B shows the deflection of the PMMA membrane as a function of the radius for different membrane thicknesses;

FIG. 39 provides an exemplary scheme of the experimental setup to test the valve with pressure driven flow and the flow rate across the valve measured with a flow sensor;

FIG. 40 provides an exemplary scheme of the sensing and the actuating circuit connected to an Arduino;

FIG. 41 provides results of an experiment measuring the flow rate across the valve as a function of the applied pressure for three different PDMS membranes;

FIG. 42 provides the results of an experiment measuring the flow rate across the valve as a function of applied pressure at the outlet;

FIG. 43A provides an exemplary on/off plot running 1000 on/off cycles, and FIG. 43B provides a plot illustrating corresponding leakage in the closed state during each cycle shown;

FIG. 44 illustrates the behavior of the valve actuated with a sinusoidal pulse width modulation (PWM) signal in the range of 27-73%;

FIG. 45A illustrates the closing of the valve with digital actuation, and FIG. 45B illustrates the closing of the valve with PWM actuation;

FIG. 46 shows leakage as a function of the applied pressure for both PMMA membranes;

FIG. 47 shows an exemplary design of a polymerase chain reaction (PCR) chip;

FIG. 48 shows custom made adapters to increase the number of valves that can be actuated at a given time;

FIG. 49 illustrates stable transitions temperature holds at each step;

FIG. 50 shows exemplary thermocycling measurements from use of a PCR chip compared to a theoretical target temperature; and

FIG. 51 shows exemplary results from detecting the PCR product of mutant DNA on the PCR chip compared to the end point results from a gold-standard qPCR machine.

### DETAILED DESCRIPTION

Aspects described herein utilize two or more filters (e.g., a double filter design) that are able to separate components of whole blood (e.g. plasma, cells, cell-free nucleic acids in circulation). The filters can be “tuned” and can be swapped out (e.g. different size pores) depending on the application needed (e.g. different targets being analyzed, such as circulating nucleic acids, or nucleic acids inside cells which require additional lysis). Conventional filters can be used. Although such filters are available on market, and they are typically not combined together. As described herein, filters can be coupled to a device for use without the need for custom fabrication. Such devices can be made out of polymer (e.g., 3D printed) or plastic (PMMA) and coupled to a standard pump (see below) without the need for special equipment.

Fluid (e.g., whole blood) can be provided to the two-filter system using any type of pump (e.g. syringe or peristaltic), and parameters can be tuned based on downstream applications (e.g. flow rate can be optimized to avoid lysis of cells if necessary). This system can process milliliter volumes of whole blood which can be coupled to downstream processes on the microliter scale, despite the differences in volume scales. The filters system can extract, for example, about 500 microliters of plasma from un-diluted whole blood without clogging. In contrast, many portable sample preparation devices are on the microfluidic scale and can only extract microliters of plasma.

In one aspect, the filter system can be coupled to a microfluidic chip for nucleic acid extraction utilizing, for example, silica magnetic beads and chaotropic salts. In this aspect, filter system can be connected to a microfluidic amplification module for nucleic acid amplification, which utilizes a small-scale thermoelectric cooler for both active heating and cooling. All microfluidic components can be fabricated in plastic (e.g., PMMA) for disposability.

The exemplary filter system can extract large volumes of components from unprocessed (undiluted) whole blood without clogging or leakage and without significant force, such as centrifugation, making sample preparation compatible with downstream assays and without requiring dilution of whole blood. The exemplary system is tunable and adaptable to different applications (e.g., tune filters and settings for extraction, and tune reagents in a nucleic acid amplification chip (i.e., lysis buffers, PCR primers and polymerases, etc.). The systems and components described herein can be used in any suitable application, including, but not limited to, separation of blood components for other medical applications (e.g., infectious disease, hematology, etc.), separation of other fluids, and integration sample prep for lab-on-chip devices. Aspects described herein can be used for any medical application requiring sample pre-processing (e.g., point-of-care or lab-based).

The exemplary filter system can utilize a 3D printed holder with grooves and an O-ring to separate the two filters. Despite the large sample volumes being processed, the whole filtration procedure can be done without clogging, and at such low pressure that a peristaltic pump can be used, allowing for point-of-care analysis. Circulating DNA is able to move through the 2-stage filter for downstream microfluidic analysis, demonstrating the low sample loss.

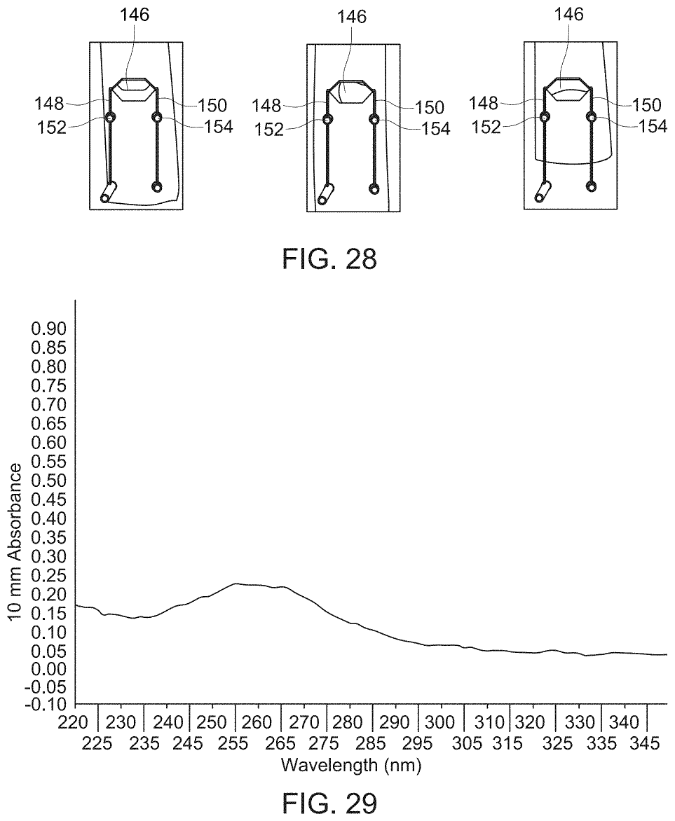

The exemplary two-membrane design system can be used with a downstream microfluidic chip with magnetic beads for isolating and concentrating DNA, followed by elution into a smaller volume for downstream analysis. In this aspect, the two-membrane design can be first integrated with a microfluidic chip capable of concentrating the sample into an even smaller (5-50 μL) volume than the original starting volumes of sample (0.5-5 mL), and then used for further analysis. In this aspect, the input volume for the two membrane design system can be, for example, about 500 μL-5 mL and the output from the two-membrane design system can be about 200 μL-1 mL. The input volume for the DNA purification component can be about 200 μL-1 mL and the output volume can be about 5-50 μL. An example how the microfluidic chip can concentrate the sample is shown in FIG. 28 and accompanying text.

Microfluidic analysis for use in aspects described herein can be carried out using a microfluidic design which enables valving to control fluid movement for temperature cycling and real-time detection of nucleic acids.

Both sides of an exemplary microfluidic chip can be made entirely out of plastic (poly(methyl) methacrylate (PMMA) or other plastics). Sealing can be done on one side with a cover slip of PMMA using isopropanol and heat, while sealing on the other side can done with a standard adhesive (PCR adhesive or other). The side can also be sealed with elastomer (i.e. PDMS) which is typically used in such valves. Thus, different materials can be used to fabricate devices (e.g., PDMS polymer, plastic, or adhesive).

In one aspect, the microfluidic chip contains vertical valving components with concentric circles that can be actuated by a solenoid, such that sealing with adhesive and actuating with a solenoid can withstand significant pressure build-up (10-15 bar) due to thermal build-up (this is non-obvious as it is very difficult to withstand high pressure build-ups in hard plastics without leakage). The valving components that can be pulsed to actuate different flow patterns and control overflow. The solenoids can also be pulsed with modulation to tune to different applications.

Microfluidic chips, as described herein can be of a mutually compatible design to accommodate placement of components for valving (i.e. solenoids), temperature control (heating/cooling), and real-time fluorescence detection.

In one example, DNA amplification and detection, temperature control, and real-time fluorescence detection are done in one chamber. A thermoelectric cooler and a K-type thermocouple can be used to achieve fast and successful thermal cycling. The thermocouple is placed under the chip and on top of the thermoelectric cooler, such that the temperature can be measured sufficiently accurately enough to perform PCR (which is a highly sensitive and temperature-specific laboratory assay). This design does not require any use of thermocouple placed directly inside the chamber. The size and thickness of the plastic is suitable for appropriate heat transfer.

On the other side of the chip is a one-sided fluorescence detection system for DNA amplification and real-time DNA detection. In contrast, conventional designs for thermocycling require elements on both sides of the chip, and same for fluorescence detection. In this aspect, the amplification/detection elements are spatially compatible with the components for the valving. The system can utilize laboratory techniques such as PCR, isothermal amplification, and other traditional laboratory assays.

Aspects described herein provide a blood sample preparation system having a filter component comprising at least a first filter and a second filter for preparing a blood sample where each filter has a different pore size, and a microfluidic amplification component for amplification of nucleic acid from the blood sample.

The term “amplification component” refers to a device or apparatus capable of amplifying (i.e., increasing in quantity) or modifying nucleic acid (i.e., changing one or more nucleotides) using any suitable mechanism (e.g., PCR, CRISPR). In one aspect, the amplification component can be coupled with or connected to the filter component, as described herein, such that a filtered blood sample can be directed through channels, for example, to the amplification component following filtering.

The term “filter” as used herein refers to device capable of separating components in a medium such as a liquid. For example, filters can be used to separate components of complex biological fluids such as blood. Such filters can be made of any suitable material, as described herein, or shape so as to adapt to use in conventional or custom filtration components.

In one aspect, the volume of the blood sample is greater than about 100 pl. In another aspect, the microfluidic amplification component is in fluid connection with the filter component. In yet another aspect, the DNA purification component is in fluid connection with the filter component and the microfluidic amplification component.

Optionally, the blood sample preparation system can have a DNA purification component (e.g., magnetic beads) for further nucleic acid purification following, for example, use of the first and second filters to separate components of blood for further analysis and processing. The term “DNA purification component” refers to a device or apparatus capable of purifying nucleic acid (i.e., removing components other than nucleic acid) through suitable methods (e.g., column chromatography, magnetic beads, PCR). For example, DNA or another nucleic acid can selectively bind to magnetic beads and be separated from a fluid.

In one aspect, the blood sample volume into the filter component is about 500 μL-5 mL and the output volume from the filter component is about 200 μL-1 mL. The fluid input volume into the DNA purification component can be about 200 μL-1 mL. This input volume can be reduced during DNA purification by binding DNA to a purification component (e.g., magnetic beads) and eluting the DNA in a smaller volume (e.g., 5-50 See, e.g., FIG. 28 and accompanying disclosure provided herein.

The filter component can include an inlet component having an inlet port for receiving a blood sample, an outlet component having an outlet port for releasing a filtered blood sample, and a filter support for retaining the first filter and second filter.

As described herein, the filter membranes can have a pore size for excluding blood components (e.g., cells). In one aspect, the pore size of the first filter is larger than the pore size of the second filter. In this aspect, the first filter can remove larger components of blood while the second filter can remove smaller blood components. In addition to the advantages described herein, use of two filters can more efficiently filter blood components, requires smaller volumes, and reduces clogging of filtering devices.

As described herein, the filter component can have a first side and a second side. The first filter can be retained on the first side of the filter component and the second filter can be retained on the second side of the filter component. Optionally, a microchannel is disposed between the first filter and the second filter.

The pore size of the filters can range from about 1 μm to about 25 μm. In one aspect, the pore size of the first filter and the second filter range from about 2 μm to about 6 μm. In another aspect, the pore size of the first filter is about 5 μm and the pore size of the second filter is about 3 μm. In yet another aspect, the pore size of the first filter is about 5 μm and the pore size of the second filter is about 2 μm. In a further aspect, the first filter is about 3 μm and the pore size of the second filter is about 2 μm.

Optionally, the blood sample preparation system has a pump for providing a blood sample. The pump can be any suitable pump (e.g., a syringe pump and a peristaltic pump). The flow rate of the pump can be from about 100 μL/min to about 600 μL/min.

In another aspect, the microfluidic amplification component includes a polymerase chain reaction chamber for amplifying nucleic acid.

Optionally, the microfluidic amplification component has at least two inlets for the polymerase chain reaction chamber for providing reagents to the polymerase chain reaction chamber. The microfluidic amplification component can also include a channel connecting the at least two inlets for mixing reagents. In another aspect, the microfluidic amplification component includes an outlet from the polymerase chain reaction chamber for disposal of waste products.

In one aspect, the microfluidic amplification component includes at least one valve for controlling flow of reagents to and from the polymerase chain reaction chamber. In yet another aspect, the at least one valve is a solenoid valve.

In one aspect, the solenoid valve comprises a sensing tip. In another aspect, the microfluidic amplification component further comprises an actuator (e.g., a membrane). The membrane can be made of any suitable material (e.g., PDMS and PMMA).

Optionally, microfluidic amplification component includes an extraction chamber disposed between the at least two inlets and the polymerase chain reaction chamber for extracting nucleic acid from the blood sample.

Aspects described herein also provide methods of filtering and amplifying a nucleic acid target from a blood sample by providing a blood sample to a filtering component; filtering the blood sample through a first filter and a second filter to form a filtered blood sample; and amplifying the nucleic acid target from the filtered blood sample.

The blood sample can be provided to the filtering component by a pump (e.g., a syringe pump and a peristaltic pump). The flow rate of the pump can be from about 100 μL/min to about 600 μL/min.

In another aspect, the first filter and second filter have a pore size, and the pore size of the first filter is larger than the pore size of the second filter. The pore size of the first filter and the second filter can range from about 1 μm to about 25 μm or about 2 μm to about 6 μm.

In one aspect, the pore size of the first filter is about 5 μm and the pore size of the second filter is about 3 μm. In another aspect, the pore size of the first filter is about 5 μm and the pore size of the second filter is about 2 μm. In yet another aspect, the pore size of the first filter is about 3 μm and the pore size of the second filter is about 2 μm.

Further aspects provide methods of filtering a blood sample and amplifying a nucleic acid target from the blood sample by providing a blood sample to a filtering component; filtering the blood sample through a first filter and a second filter to form a filtered blood sample; purifying nucleic acid from the filtered blood sample to form a purified nucleic acid sample; and amplifying the nucleic acid target from the purified nucleic acid sample. The nucleic acid can be purified from the blood sample with, for example, magnetic beads.

In this aspect, a blood sample volume into the filter component is about 500 μL-5 mL and a fluid output volume from the filter component is about 200 μL-1 mL. In another aspect, an output volume after purifying nucleic acid from the filtered blood sample is about 5-50 μL.

### EXAMPLES

### Example 1

### Sample Preparation—Dual Membrane Filtration for Plasma Extraction

Exemplary devices and methods for blood plasma extraction with the specific goal of subsequent cfDNA (cell-free DNA) isolation and analysis utilizing the following features are presented below:
Large (ml) volume of sample processed
Detection of very low level of analytes (e.g., cfDNA)
Lysis-free design (e.g., white blood cells (WBCs), to avoid genomic DNA contamination)
High yield (volume) in a reasonable amount of time
Easy integration with downstream applications (e.g. a cfDNA purification stage or a direct PCR stage)

For this reason, an exemplary multi-stage filter device that utilizes parallel membranes of subsequently smaller pore sizes was selected. Together, these membranes eliminate the cellular components of blood, step by step. In this way, a continuous filtration can be achieved, and pure plasma can be collected in a continuous manner while avoiding fast clogging that often results in typical single membrane devices. At the same time, the multi-stage filter achieves faster separation time than sedimentation, and creates a highly purified plasma product.

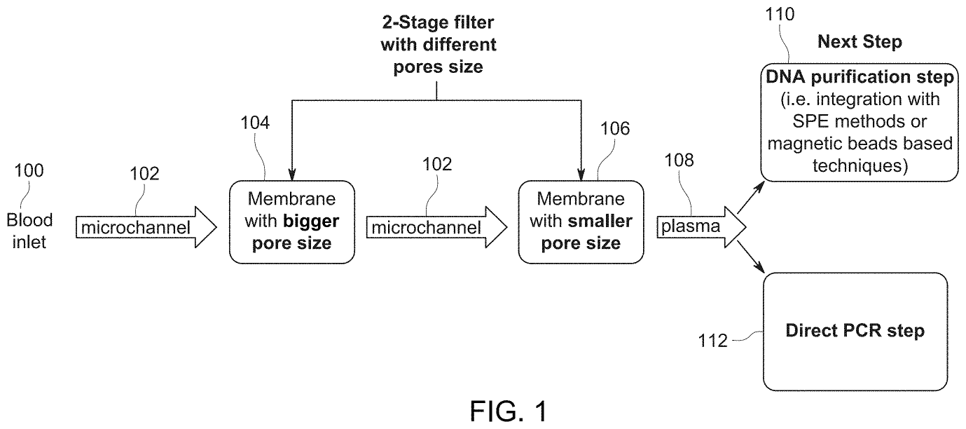

An exemplary schematic illustration of the two-stage filter is presented in FIG. 1. In reference to FIG. 1, a blood sample can flow through blood inlet 100 into microchannel 102 into larger pore membrane 104 through a second microchannel 102 into smaller pore membrane 106. Following two-staged filtering through larger pore membrane 104 and smaller pore membrane 106, the filtered blood plasma 108 can be sent to DNA purification system 110 and to Direct PCR system 112 for further analysis as described herein.

Membrane materials and pores sizes can be selected based on the desired application. In one aspect, the material can be selected to facilitate analysis of the cell free DNA contained in blood plasma. Thus, a material, such as polycarbonate, with low nucleic acid binding can be chosen to avoid loss of the cfDNA analyte during the filtration process. Polycarbonate has minimal protein and nucleic acid binding.

The pore size can be selected to specifically accommodate the diameters of both red blood cells (RBCs) and WBCs. RBCs range from 6-7 μm and WBCs range from 7-25 μm. Furthermore, in literature it has been demonstrated that RBCs, due to their deformability, can pass through a pore size of 5 μm. For this reason, membranes with a pore size starting from 5 μm can be used (e.g., Whatman® Nuclepore Track-Etched, purchased Sigma-Aldrich®, with pore sizes ranging from 5 μm to 2 μm).

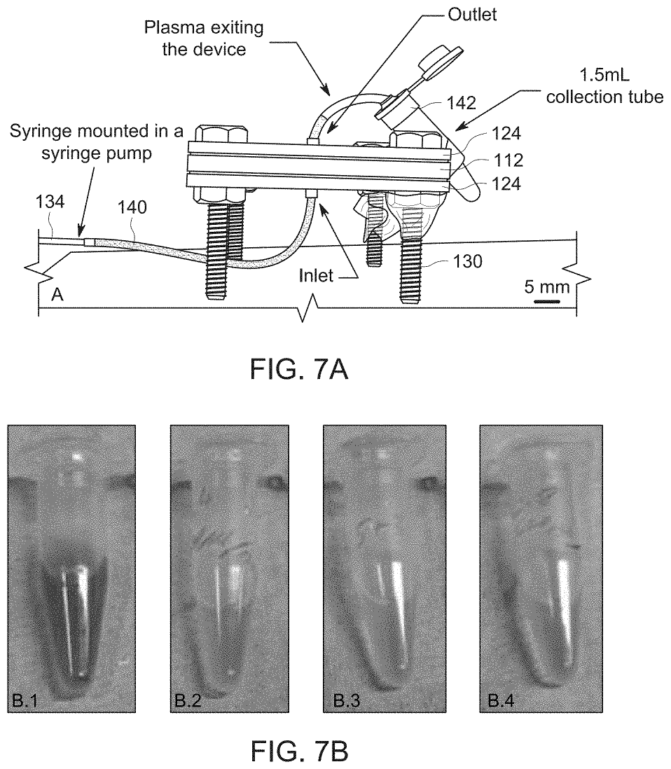

In another aspect, two different configurations of filter-based devices can be selected: cross-flow filtration or dead-end filtration. Dead-end filtration can be used to avoid loss of analytes, and thus selecting a flow direction that is perpendicular to the membranes and in the same direction of the collection channel could help cfDNA recovery. In addition, there are three different orientations that can be paired with dead-end filtration. Depending on the position of the membrane with respect to the direction of fluid flow, one can choose between a “membrane-on-bottom” configuration, “membrane-on-side” configuration and “membrane-on-top”configuration (e.g., FIGS. 7A-10).

The membrane-on-bottom configuration allows the sedimentation of cells to occur simultaneously during the filtration process, since fluid flow occurs in the same direction as gravitational sedimentation. This could lead to faster clogging of the membrane since there are two forces (filtration and sedimentation) working in concert to pull the fluid toward the membrane. Membrane-on-side and membrane-on-top configurations allow for maximize filtration capacity by redirecting the sedimentation toward the bottom of the filter chamber, maximizing the time before clogging can happen. In particular, the membrane-on-top configuration has been shown to achieve the best results in term of membrane area available without clogging for the longest amount of time.

Filtration Component Design:

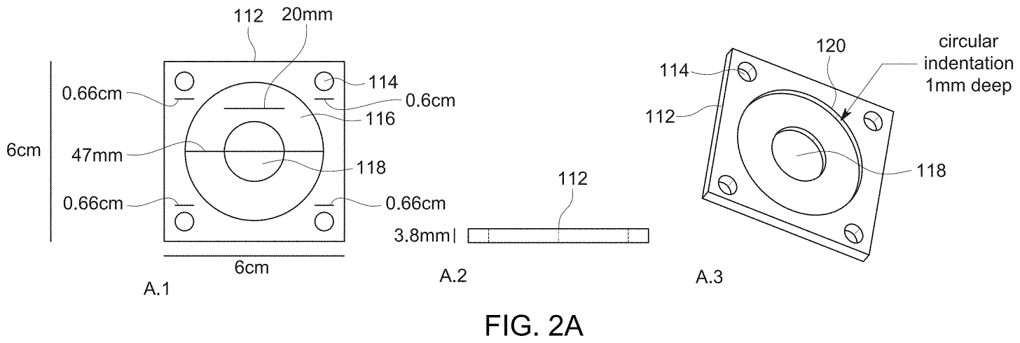

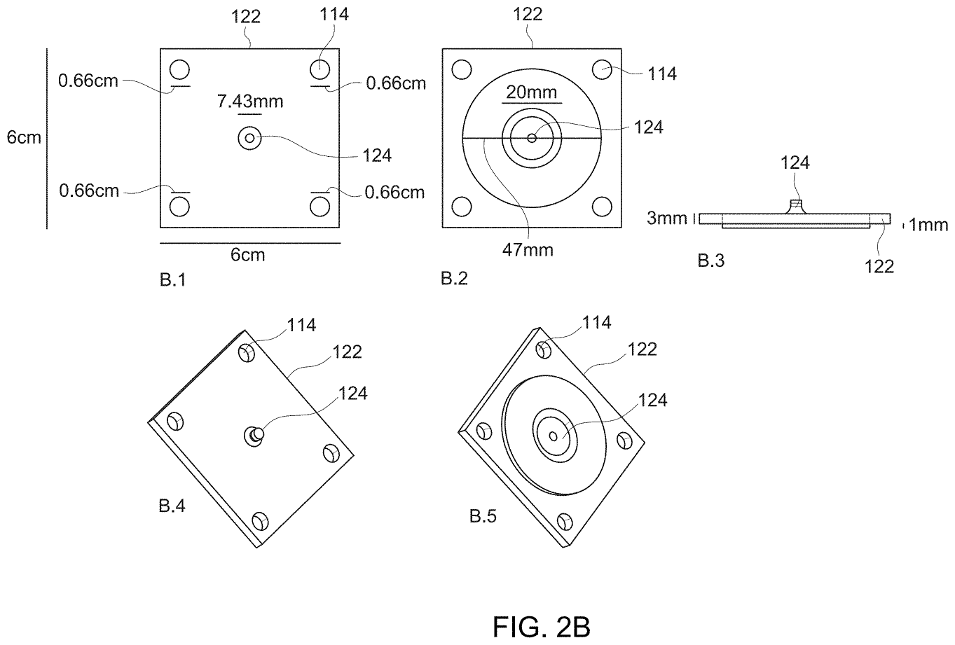

In reference to FIGS. 2A and 2B, an exemplary 3D printed membrane-on-membrane (MOM) structure was designed and tested. In this example, a PMMA+3D printed polymer was used to permit more sophisticated geometry for the filter. This iteration of the device is compatible with a continuous flow fluid control mechanism and was able to contain about 1.7 ml of fluid inside (i.e. designed with a “void volume” of ˜1700 mm3) as well as the 2 membranes.

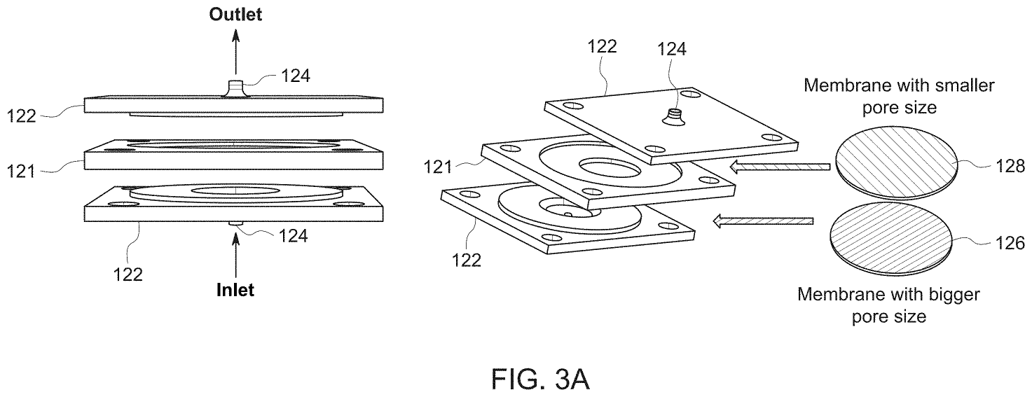

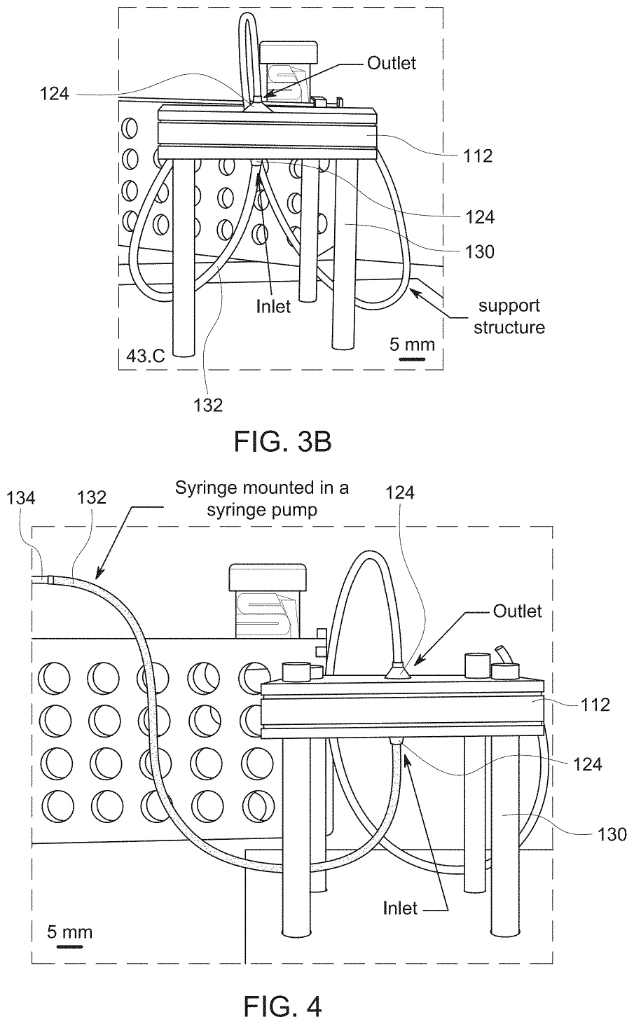

The structure from 3 different parts that can be assembled together. The symmetrical geometry of the device allows for flow from both the top and from the bottom. FIGS. 2A and 2B show the design of each part of the MOM device 112 in detail, while FIG. 3 shows the complete MOM device 112 and the final assembled MOM device 112.

The top side of the middle layer of the MOM device 112 is shown in FIG. 2A.1 having post holes 112. Middle layer 121 of the MOM device 112 can accommodate two membranes—one on top and one on the bottom. Middle layer 121 of the MOM device 112 has a symmetrical design, with a first circular indentation 116 of 1.5 mm where a 47 mm diameter polycarbonate membranes can sit. In addition, a hole 118 of 20 mm in diameter allows the flow of the liquid through the membranes in a vertical direction. FIG. 2A.2 provides a side view of the middle layer 121 of the MOM device 112.

FIG. 2A.3 shows the bottom side of the middle layer 121 of the MOM device 112 with second circular indentation 120 for accommodating a second membrane.

FIG. 2B shows the inlet/outlet layers 122 of MOM device 112. A top view (2B.1), bottom view (2B.2), side view (2B.3), angled bottom view (2B.4) and angled bottom view (2B.5) are shown. The top and the bottom inlet/outlet layers 122 can be identical to each other and served as inlet and outlet collection chambers when integrated with the middle layer of the MOM device 112. Tubes (not shown) can be used to connect inlet/outlet layers 122 with middle layer of the MOM device 112 through holes 114. Inlet/outlet 124 can be connected to other aspects described herein and permit fluid to flow into and out of MOM device 112.

Inlet/outlet 124 are designed as a cone shape structure on one side, to match with a 1.5 mm in diameter tube, and an empty cone-shape on the other side. This last feature allows a symmetrical filling from the inlet as well as a symmetrical liquid collection toward the outlet. The exemplary structure was assembled with two polycarbonate membranes with 5 μm and 3 μm pore size respectively.

The four holes 114 at the corners, 0.66 cm in diameter, can permit correct alignment during the assembly of the device and integrate a support structure for the device.

FIG. 3A shows side views of an exemplary assembly of MOM device 112 having inlet/outlet layers 122 above and below middle layer 121 with inlet/outlet 124 shown and the top and bottom of MOM device 112. FIGS. 3B and 4 provide a photographs of an exemplary assembled MOM device 112 having inlet/outlet layers 122 above and below middle layer 121 with inlet/outlet 124 shown and the top and bottom of MOM device 112. Support structures 130 are shown for use in holes 114. Tubing 132 is shown connected to inlet/outlet 124.

The structure was tested with a flow from the bottom to the top with tinted water. A support structure made by 4 plastic poles was created to support the device. 4 ml of water was loaded into the syringe, mounted in the syringe pump, and connected to the inlet tube of the device. The outlet was connected with a 1.5 mm inner diameter tube to collect fluid. The chosen flow rate to test the device was 200 μL/min. FIG. 4 shows the device in operation with inlet 124 connected to syringe pump 134 by tubing filled with a red colored blood sample and a clear fluid exiting the outlet 124 after passing through the exemplary dual-membrane MOM device 112.

It took the fluid shown in FIG. 4 approximately 9 minutes after entering the device to appear in the outlet. The void volume inside of the device contained about 1.7 ml of liquid with flow rate of 200 μL/min, confirming that there was no leakage in the structure. This was also confirmed by visual observation.

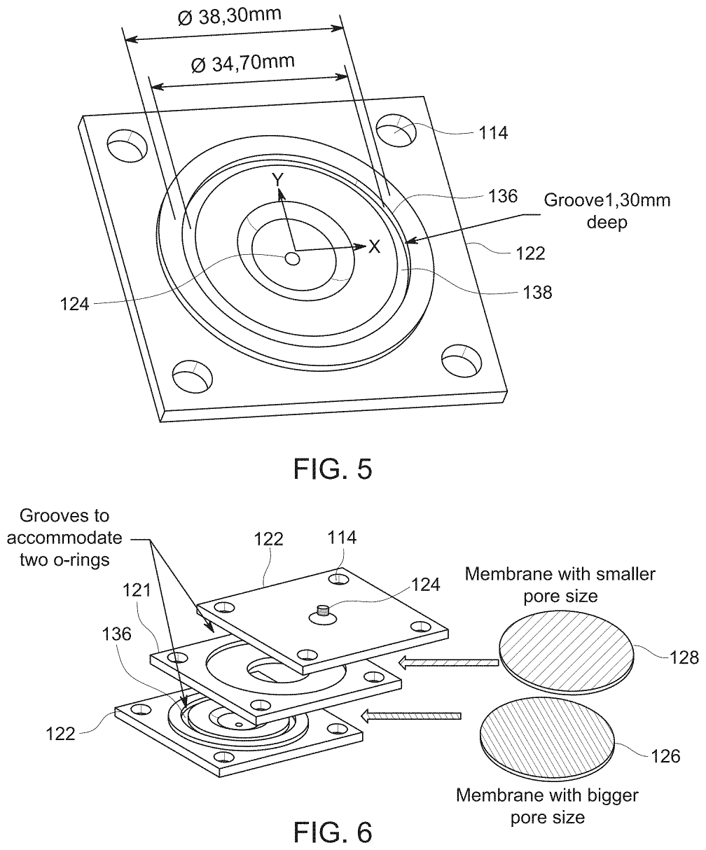

In order to prevent any possible leakage, an optional groove 136 accommodating an optional O-ring 138 was created in both the top and bottom parts as shown in FIG. 5. An O-ring is a mechanical gasket made by an elastomer loop that when seated and compressed in a groove creates a sealing between the two parts that are compressed. FIG. 5 shows the modified design for the bottom and top part with the implementation of a groove 136 to accommodate the O-ring 138. In particular, a Viton® 60 FDA O-ring (34.65 mm inner diameter, 38.21 outside diameter, 1.78 mm thick) was used in this case, and the designed groove had a depth of around 70% of its thickness for a correct final sealing.

This exemplary was tested with clinical blood samples. A schematic of the complete structure of the exemplary device including groove 136 is presented in FIG. 6.

As described herein, blood samples can be diluted in accordance with a desired dilution factor, a choice of membranes and pore sizes, and a flow rate in order to obtain a large enough quantity and a pure enough sample of plasma, from which it will be possible to conduct studies relating to analyzing circulating fragments of, for example, circulating free DNA (cfDNA).

For example, PBS (phosphate buffered saline) can be used to dilute the blood and undiluted human whole blood and samples diluted in a 1:8 ratio (i.e., 1 part of whole blood and 7 parts of PBS) can be tested.

In another example, a flow rate of 100 μl/min and two different circular polycarbonate membranes, each 47 mm in diameter, one with 5 μm pores and the other with 3 μm pores, can be used to optimize the dilution factor. In addition, at least 500 μl of plasma was collected, to have enough sample volume to perform cfDNA detection studies even with the highest dilution.

As shown in FIG. 7A, exemplary blood samples were loaded in a 5 ml syringe mounted in a syringe pump 134. The syringe adapter was linked to the inlet of the device through a 1.5 mm inner diameter tube 140. The same type of tubing was used to link the output with a 1.5 ml collection test tube for output collection into collection tube 142.

The exemplary device was tested with 4 human blood samples with different dilution conditions: 1) whole blood (no dilution), 2) 1:4 dilution with PBS, 3) 1:6 dilution with PBS, and 4) 1:8 dilution with PBS. Once the sample entered the device, due to the chosen design, approximately 18 minutes was needed to start collecting the output at a flow rate of 100 μl/min. Output from the four conditions was collected for at least 5 minutes after appearing at the outlet. The collected outputs are shown in FIG. 7B (B.1—whole blood, B.2—1:4 dilution in phosphate buffered saline (PBS), B.3—1:6 dilution in PBS, B.4—1:8 dilution in PBS).

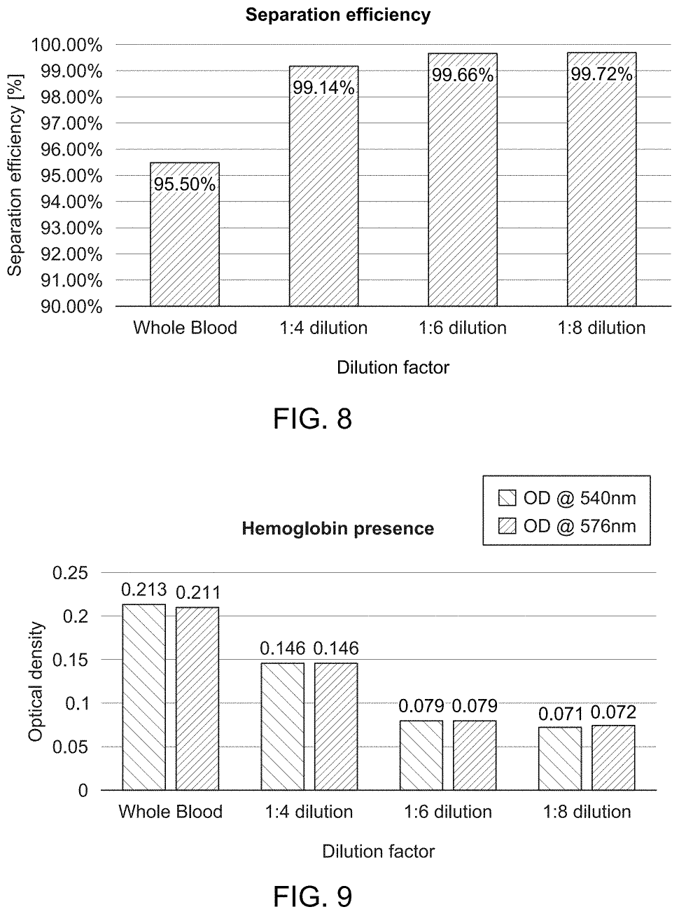

A hemocytometer was used to calculate the separation efficiency, as shown in FIG. 8. As shown in FIG. 8, the separation efficiency increased with the dilution factor—from 95.5% in whole blood to 99.72% in the 1:8 dilution sample. Further, the quality of the extracted plasma, characterized by the degree of hemolysis, was quantified using a spectrophotometer. The absorbance levels for the various output samples are shown for two specific wavelengths (540 nm and 576 nm) related to the presence of hemoglobin. The results from this are shown in FIG. 9. As shown in FIG. 9, the presence of hemoglobin decreased at two optical density readings (540 nanometers (nm) and 576 nm) as the dilution factor increased.

In addition, the flow rate was analyzed. As discussed above, a flow rate of 100 μl/min was the fixed value chosen to start the experiments, since it was likely low enough to avoid hemolysis and most useful to characterize the optimal dilution factor. Thus, in order to characterize different flow rates, a combination of two membranes with 5 μm and 3 μm pores size with a 1:6 dilution factor was used.

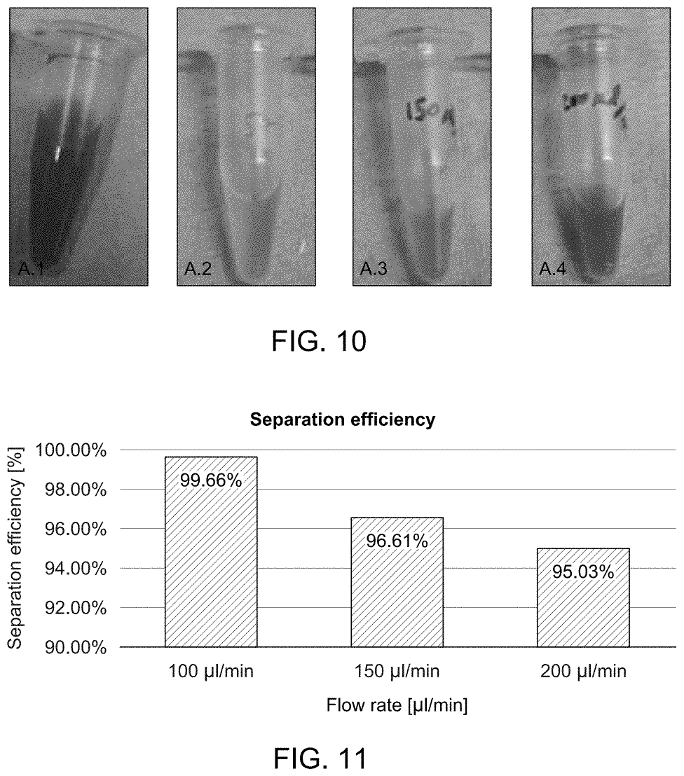

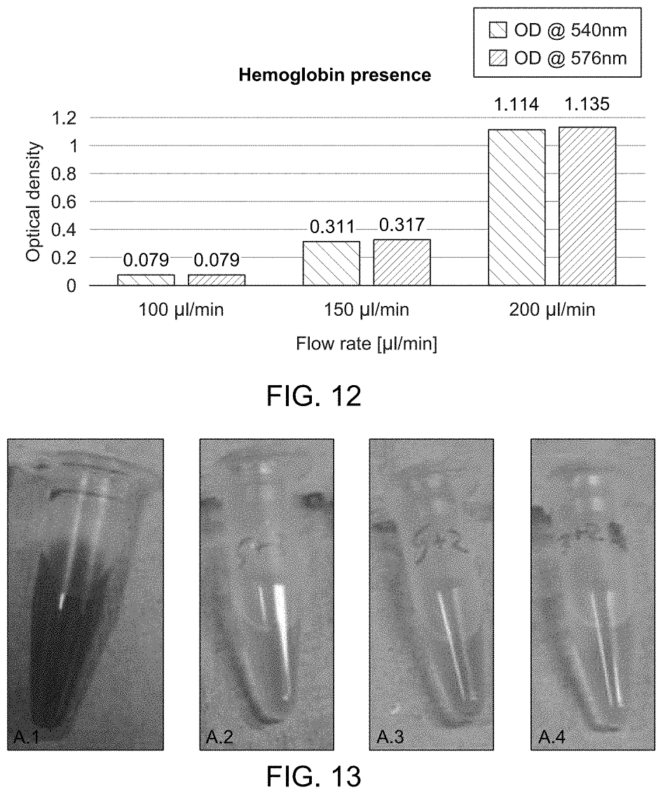

In one aspect, a higher flow rate can be used to reduce the time needed for plasma collection which could be important in a point-of-care (POC) device. Specifically, the device was tested with three different flow rates: 1) 100 μl/min 2) 150 μl/min and 3) 200 μl/min. The experimental setup was the same as the previous set of experiments. The quality of the plasma outputs from the different flow rates were assessed again through a hemocytometer, to study the separation efficiency, and with a spectrophotometer, to study the absorbance at Hemoglobin related peaks. The output collection of the samples exiting the device related to the 150 μl/min and 200 μl/min flow rates started after around 12 minutes and 9 minutes, respectively. The collected outputs are shown in FIG. 10 while separation efficiency and absorbance studies for each collected sample are shown in FIG. 11 and FIG. 12.

As shown in FIG. 10, the lower flow resulted in a clearer collection fluid (A.1—whole blood, A.2—100 μl/min, A.3—150 μl/min, A.4—200 μl/min). Likewise, separation efficiency decreased as flow rate increased (FIG. 11) and hemoglobin presence increased as flow rate increased (FIG. 12).

Membranes were tested to determine which membrane provides the best performance for this exemplary. Three polycarbonate membranes with different pores size (5 μm, 3 μm and 2 μm) were chosen to be tested in the plasma separation process.

The exemplary fixed configuration described above was used to optimize the dilution factor and flow rate used a two-membrane filtration configuration with polycarbonate membranes of 5 μm for the first filter membrane and 3 μm for the second filter membrane. Two addition configurations, 5 μm+2 μm, and 3 μm+2 μm, were also compared.

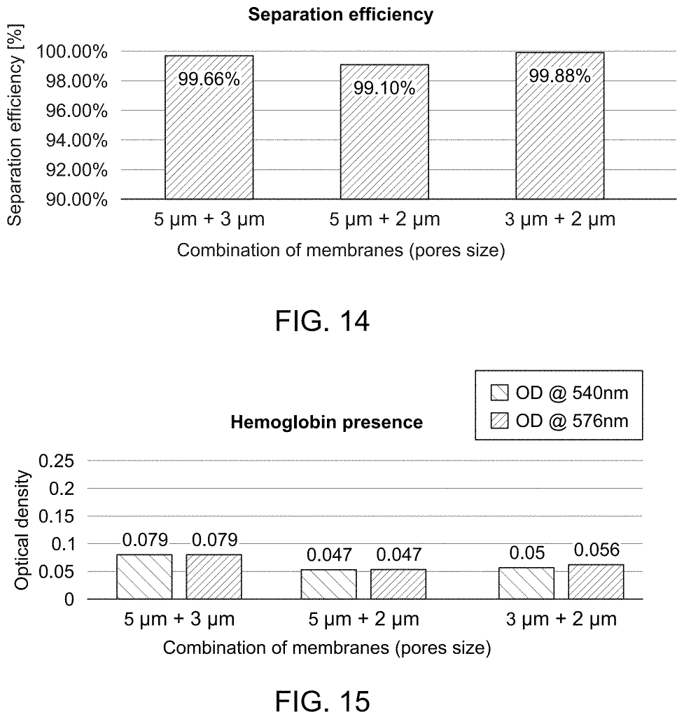

The experimental setup remained the same and the and the previously described flow rate of 100 μL/min and dilution factor of 1:6 were used. The collected samples are presented in FIG. 13, while FIGS. 14 and 15 show the values related to the separation efficiency and presence of hemoglobin.

As shown in FIG. 13, the appearance of three filtered plasma samples was approximately the same compared to whole blood (A.1—whole blood, A.2—5 μm+3 μm, A.3—5 μm+2 μm, A.4—3 μm+2 μm). Separation efficiency (FIG. 14) and hemoglobin presence (FIG. 15) was approximately the same.

As described above, the quality of the extracted plasma was assessed in terms of separation efficiency and hemoglobin level to “tune” the filter component and optimize the dilution factor, flow rate and membrane pore size combinations. The resulting purified sample was then analyzed for the presence of cfDNA.

To simulate the presence of cfDNA, a short fragment (180 bp) of the human MSTN1 gene was designed using Macvector, and synthesized by Integrated DNA Technologies (IDT®). The gene was amplified using the polymerase chain reaction (PCR) and purified with QIAquick PCR Purification Kit, purchased from Qiagen®, before being spiked in diluted blood samples. A set of primers (forward primer 5′-TTG GCT CAA ACA ACC TGA ATC C-3′ with a Tm=55.9° C. and reverse primer 5′-TTG GCT CAA ACA ACC TGA ATC C-3′ with a Tm=54.4° C.) were designed using Macvector and ordered from IDT to amplify a region of the MSTN1 synthetic DNA fragment by PCR (PCR size of 120 bp).

The gene was amplified and spiked in 5 whole blood samples at the following concentrations: 1) 25 ng/ml, 2) 250 ng/ml, 3) 500 ng/ml, 4) 750 ng/ml and 5) 1000 ng/ml. These concentrations were selected to cover a reasonable range of concentrations in which one would find cfDNA fragments in cancer patients.

The spiked human blood samples were each diluted 1:6 with PBS to a final volume of 3 ml each. The specific samples composition is described in the following list:
Sample 1: 2.5 ml PBS+0.5 ml whole blood previously spiked at 25 ng/ml
   →4.16 ng/ml final concentration of MSTN1 gene in sample 1
Sample 2: 2.5 ml PBS+0.5 ml whole blood previously spiked at 250 ng/ml
   →41.66 ng/ml final concentration of MSTN1 gene in sample 2
Sample 3: 2.5 ml PBS+0.5 ml whole blood previously spiked at 500 ng/ml
   →83.33 ng/ml final concentration of MSTN1 gene in sample 3
Sample 4: 2.5 ml PBS+0.5 ml whole blood previously spiked at 750 ng/ml
   125 ng/ml final concentration of MSTN1 gene in sample 4
Sample 5: 2.5 ml PBS+0.5 ml whole blood previously spiked at 1000 ng/ml
   166.66 ng/ml final concentration of MSTN1 gene in sample 5

The filtration device described above was used to collect 500 μl of plasma from each of the above samples. Five control samples (C.1-C.5) of the same spiked concentrations and dilution factors were subject to traditional plasma extraction by centrifugation using a standard bench-top centrifuge, Sorvall® Legend® RT, at 2350 RPM at 4° C. for 10 minutes.

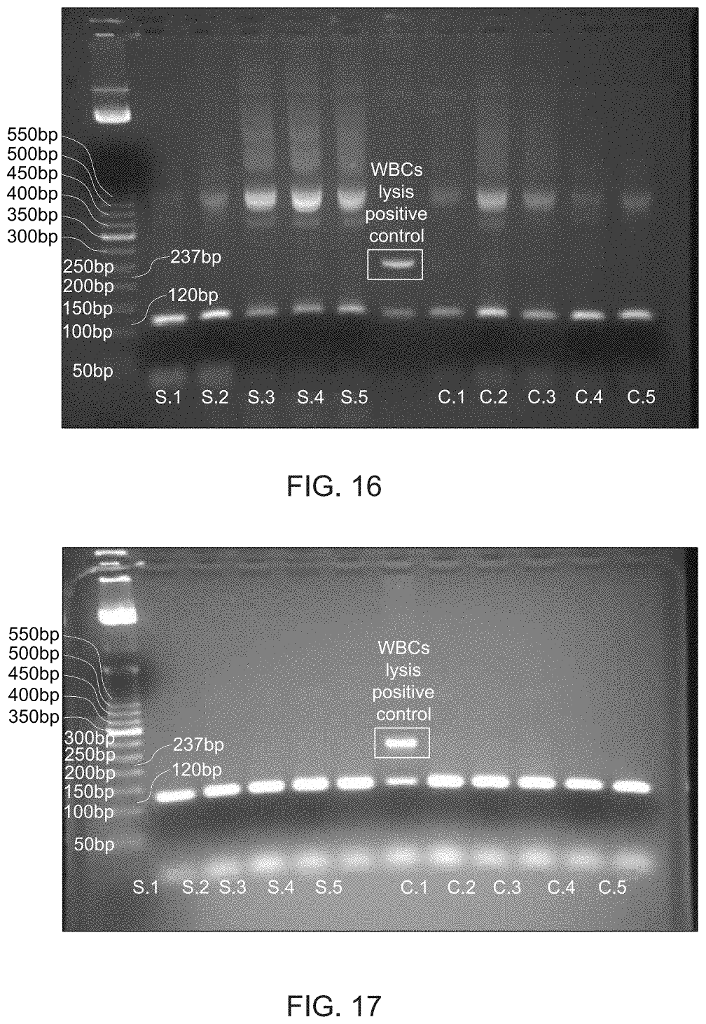

In a first experiment, once plasma was extracted for each case, a direct PCR of the collected samples was performed by using a Phusion Blood Direct PCR kit, purchased from ThermoFisher Scientific®, using the set of primers described previously. Moreover, an additional set of universal control primers was added to each reaction mixture. This set of control primers came as part of the Phusion Blood Direct PCR kit and is able to amplify a 237 bp fragment of the SOX21 gene housekeeping gene (derived from the Jurkat cell line), which is indicative of the presence of contamination mammalian genomic DNA and thus WBC lysis. A positive control for WBCs lysis (containing Jurkat genomic DNA) was prepared and used as a DNA control sample for lysis. The thermocycler protocol started with an initial denaturation (1 min at 95° C.), followed by 35 cycles including denaturation (1 second at 98° C.), Annealing (5 seconds at 60° C.) and extension (15 seconds at 72° C.) to end with a final extension step (1 minutes at 72° C.). After PCR the samples were loaded in a 2% agarose gel to perform gel electrophoresis, run at 140 V for ˜45 minutes, in order to check if it is possible to qualitatively detect the spiked fragments. The results of the PCR products after running in the gel are shown in FIG. 16.

FIG. 16 confirms the detection of the spiked MSTN1 fragments for all the tested concentrations after plasma extraction. In particular, the figure shows minimal differences between the detection of DNA from the plasma samples extracted by the device and the plasma collected via standard bench top centrifugation. This means that the quality of the plasma extracted by the built device is comparable with the plasma collected by centrifugation, validating the exemplary two-membrane filtration system. Moreover, minimal lysis of WBCs was present for all cases (FIG. 16), as indicated by the faint band running at 237 bp as compared to the strong band in the genomic DNA control sample. Furthermore, despite a slight presence, it did not interfere with the detection of rare fragments of circulating MSTN1 DNA in the plasma, thus validating the use of this plasma extraction device for subsequent cfDNA studies (in addition to the separation efficiency and hemoglobin level already discussed).

The filtration of a whole blood samples using the exemplary device can be used as a replacement for standard purification via centrifugation resulting in a lower cost and less time-consuming methodology.

In another aspect, non-specific bands can be reduced or eliminated as described. To eliminate non-specific bands, a DNA purification step following plasma extraction was performed, prior to PCR. Plasma extraction of the five samples using the micro device and the five controls using standard centrifugation was repeated, as described previously. This time, however, instead of doing direct PCR from plasma using the Phusion Blood Direct kit, cfDNA was first extracted from the plasma using the QIAamp Circulating Nucleic Acid Kit, purchased from Qiagen®. After cfDNA extraction, PCR was performed from the purified samples and from a prepared positive control for WBCs lysis (the same used in the previous experiment, containing Jurkat genomic DNA), using the Promega™ PCR Master Mix. The thermocycler protocol this time started with an initial denaturation (2 min at 95° C.), followed by 30 cycles including denaturation (30 seconds at 94° C.), Annealing (30 seconds at 60° C.) and extension (30 seconds at 72° C.) to end with a final extension step (5 minutes at 72° C.), as indicated in the kit instructions.

After PCR, all the samples were loaded in a 2% agarose gel run at 140 V for about 45 minutes. The results are shown in FIG. 17.

From the figure, non-specific bands are no longer present. Additionally, there is very minimal WBC lysis, if any, and thus that there is no genomic background DNA contaminating the sample. Thus, a purification step, using, for example, magnetic beads-based techniques, can be integrated with the exemplary two-membrane filtration component described.

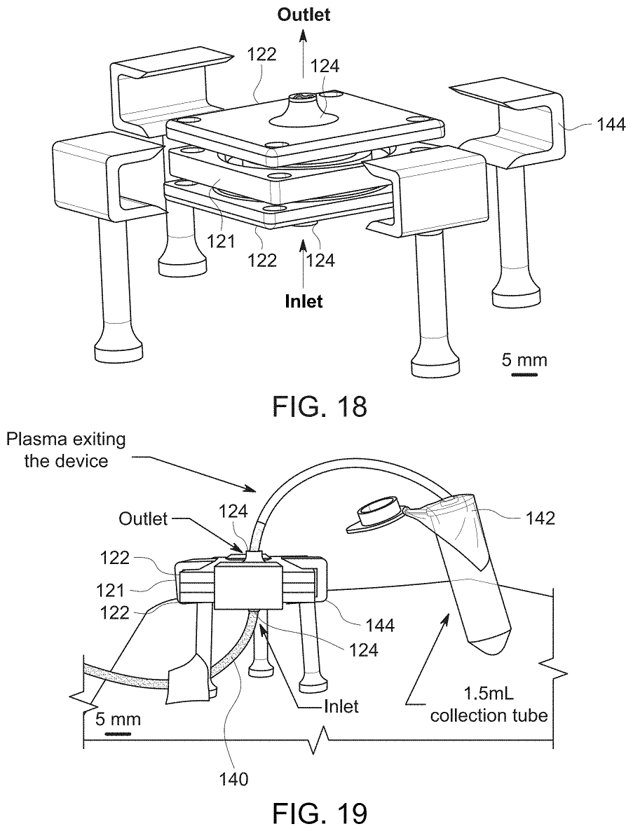

In this aspect, a device with flow rate of 100 μl/min was used to design an alternative structure with a smaller void volume, with the intent of achieving plasma separation in around 6 minutes, 3× faster than that of the previously described device. A new smaller structure was therefore designed with a geometry almost identical to the first one, but with smaller dimensions. The circular opening was reduced from 20 mm to 15 mm in diameter and the final structure, shown in FIG. 18, was designed to have a “void volume of ˜650 mm³. Moreover, 4 clips 144 were printed to close the device and to support it, to avoid using screws and nuts as in the larger prototype. This time a Silicone 70 durometer O-ring (I.D. 18.72 mm, O.D. 23.96 mm, 2.62 mm) was used. An exemplary structure is shown in FIG. 18.

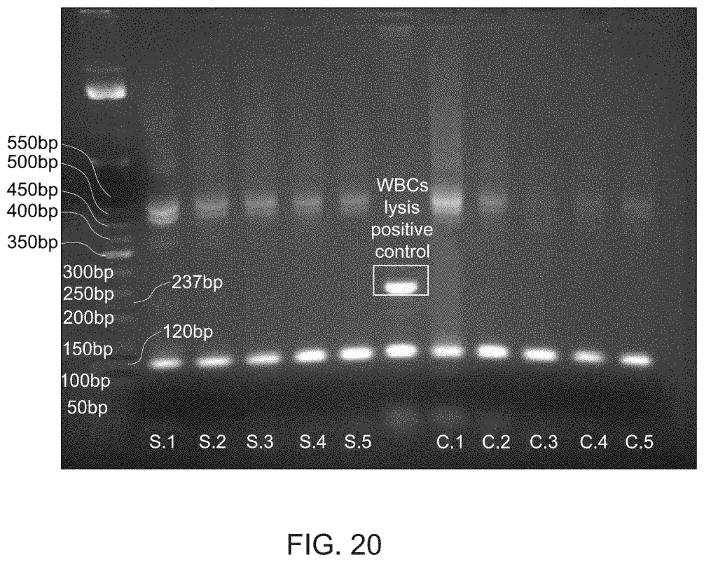

The alternative device was similarly characterized in terms of separation efficiency and plasma purity (data not shown), and was tested to see if it was possible to decrease extraction time while still detecting with the same capacity the presence of circulating MSTN1 fragments (FIG. 20). Specifically, 5 diluted samples (1:6 dilution factor) with the same concentrations of spiked MSTN1 gene used previously were prepared. The flow rate used to test the device was the same used for the bigger version, 100 μl/min, and the 5 μm+3 μm combination of membranes was also maintained. The polycarbonate membranes were cut with a laser cutter (Universal laser VersaLaser® VLS2.30, used parameters: 17% power, 90% speed) in order to match the circular indentions of 26 mm in diameter present in the bottom and top side of the middle part.

Plasma was extracted from each sample using the smaller device prototype. Output collection started after 6 minutes and 30 seconds, as expected based on the specific dimensions chosen, and plasma was collected for around 4 minutes. The separation process is visible in FIG. 19.

Though the plasma exiting the device was clear at the start of the output, it quickly turned to a deep red color soon after. The final collected samples showed absorbance values of 1,183 and 1,207 at 540 nm and 576 nm, respectively, and a separation efficiency of around 75% as determined by the hemocytometer. While these values were not as optimal as the larger prototype, but it should be noted that the used parameters for flow rate and membranes were those optimized for the larger structure and thus they don't consider the smaller available area of the membranes in this smaller prototype. Despite this, PCR studies were subsequently conducted to check the detection of the spiked MSTN1 gene.

The 5 experimental samples and the 5 control samples were again prepared with the same conditions and concentrations as described previously. Direct PCR were performed by using Phusion Blood Direct PCR kit, ThermoFisher Scientific®, and both sets of primers described before (for amplification of MSTN1 120bp segments and WBCs control lysis). The positive control for WBCs lysis containing the Jurkat gene was prepared as well. After PCR the samples were loaded in 2% agarose gel to perform gel electrophoresis for 45 minutes at 140V. The results of the PCR products running in the gel are shown in FIG. 20.

From the figure it is possible to see how, even if the quality of the plasma was lower respect the one collected from the optimized bigger device, the detection of the spiked fragments in the samples was still possible for all concentrations. Again, there were no visible differences as compared to the plasma samples extracted using standard centrifugation.

### Example 2

### Dual Membrane Filtration using Peristaltic Pump (Portable)

In the previous design and experiments, all fluids were controlled using a syringe pump which may not be optimal for POC. Thus, in another aspect the device is compatible with a portable peristaltic pump as described below. Additional testing was performed with more samples and to compare the device performance to a benchtop centrifuge.

A miniature peristaltic pump was purchased from Dolomite (Item #: 3200243). The pump is lightweight (11 g) and compact in size (30 mm×12 mm×14 mm), and thus was optimal for integration with our device. The pump was fitted with silicone tubing for attachment with the exemplary device.

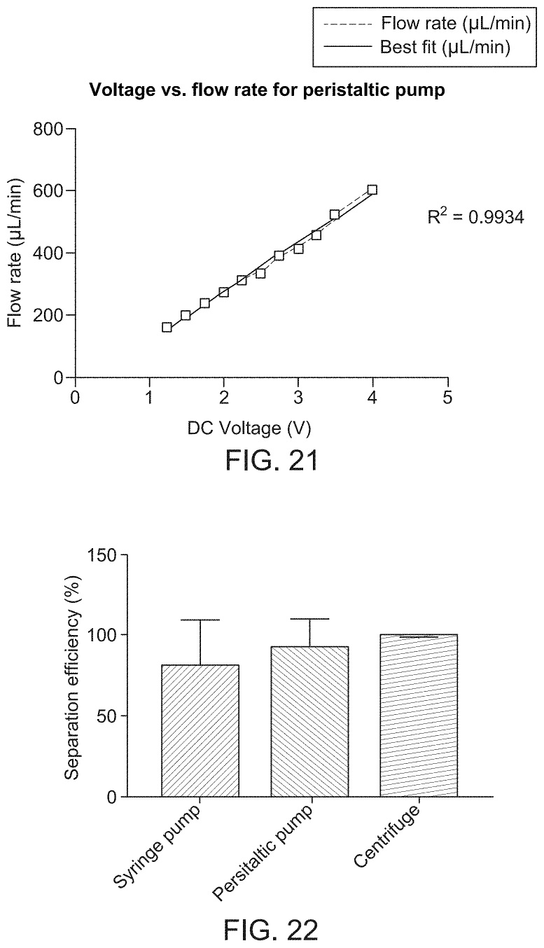

Characterization of voltage versus flow rate with the pump was first carried out to match voltage to previously designated/optimized flow rates (FIG. 21).

A flow rate of ˜0.6 V was selected to mimic flow rate of 100 μL/min optimized previously using the exemplary syringe pump as described herein. Next, comparisons between extraction efficiency using the peristaltic pump, syringe pump, and centrifuge were made (FIG. 22).

Separation efficiency values were calculated using a hemocytometer and counting the total number of cells in a diluted sample prior to and after plasma extraction. As shown in FIG. 22, an average separation efficiency of 80.23+/−29.37%, 91.59+/−18.56%, and 99.57+/−0.3791% was found for samples tested using the syringe pump, peristaltic pump, and centrifuge control, respectively. It should be noted that samples were tested anywhere from 1-10 days after sample collection, however, visible lysis was seen to begin to occur 2-3 days after sample collection. Whole blood (no dilutions) was used for all testing. Further, it should be noted that since the peristaltic pump has a voltage-driven pulsed signal, about half the volume is collected in a given time frame compared to that of the syringe pump.

One-way ANOVA followed by Tukey's multiple comparison tests were performed for the three groups using Prism. There was no statistical difference found between the three groups (p=0.2255) by one-way ANOVA. Using Tukey's multiple comparison test, the syringe pump and peristaltic pump showed no statistical difference (p=0.5783), the syringe pump and centrifuge showed no statistical difference (p=0.2007), and the peristaltic pump and centrifuge showed no statistical difference (p=0.7601). Thus, it can be concluded that, using separation efficiency as a measure, the syringe and peristaltic pump set-ups both performed comparably to that of the centrifuge control, although the peristaltic pump had better performance compared to the syringe pump for this application.

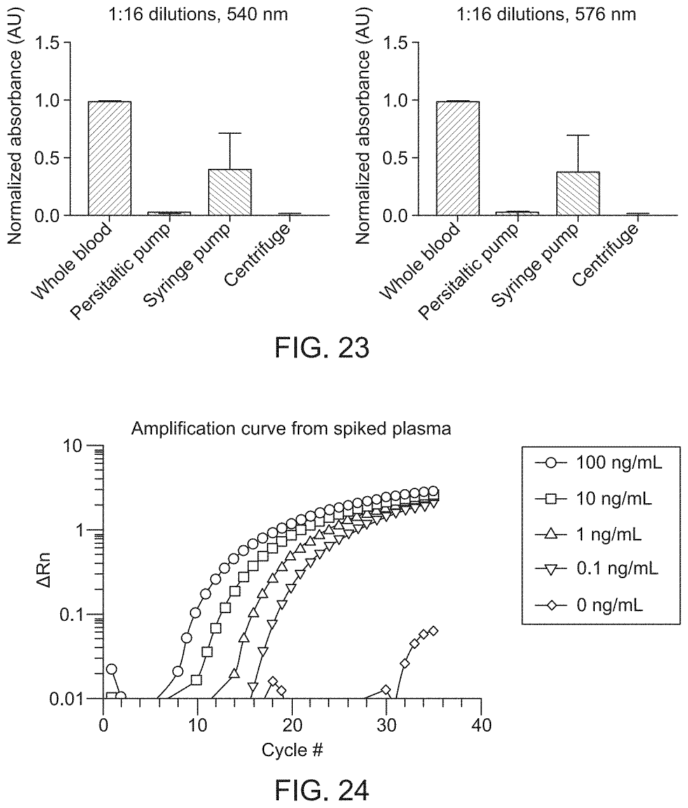

Next, the degree of hemolysis that occurred during plasma extraction was tested for each of the three different set-ups. A plate reader (BioTek) was used to measure absorbance at 540 nm (left panel) and 576 nm (right panel) (FIG. 23).

1:16 dilutions of sample were made to comply within the absorbance levels of the plate reader. At 540 nm, the mean normalized values were found to be 0.9753+/−0.01296, 0.01386+/−0.007945, 0.3887+/−0.3203, 0.007533+/−00518 (A.U) for whole blood, peristaltic pump, syringe pump, and centrifuge control, respectively. Using One-way ANOVA followed by Tukey's multiple comparisons test, a statistically significant difference was found amongst all four groups (p<0.0001).

A significant difference was found between whole blood vs. peristaltic pump (p<0.0001), whole blood vs. syringe pump (p=0.0002), and whole blood vs. centrifuge (p<0.0001). No significant difference was found between the peristaltic pump and centrifuge control (p=0.9856), syringe pump and centrifuge control (p=0.4691), and peristaltic pump and syringe pump (p=0.6795). At 576 nm, similar results were found. The mean normalized absorbance values at this wavelength were found to be 0.9761+/−0.01246, 0.01698+/−0.01271, 0.3643 +/−0.3263, 0.007135+/−0.003941 (A.U.) for whole blood, peristaltic pump, syringe pump, and centrifuge control, respectively.

Using One-way ANOVA followed by Tukey's multiple comparison test, a statistically significant difference was found amongst all four groups (p<0.0001). A significant difference was found between whole blood vs. peristaltic pump (p=0.0001), whole blood vs. syringe pump (p=0.0008), and whole blood vs. centrifuge control (p<0.0001). No significant difference was found between the peristaltic pump and centrifuge (p=0.8966), syringe pump and centrifuge (p=0.4679), and peristaltic pump and syringe pump (p=0.8666). Once again, it can be concluded that, while both the peristaltic pump and syringe pump set-ups are comparable to the centrifuge control for plasma extraction, the peristaltic pump performs slightly better.

Additional testing was performed on spiked whole blood samples of cfDNA fragments to test percent recovery after passage through the exemplary plasma extraction device for a quantitative assessment (FIG. 24). For this experiment, testing DNA concentrations ranged from 1000 ng/ml to 0.01 ng/ml to mimic concentrations of cfDNA. The amount of DNA recovered was quantified after extraction by performing direct qPCR of the resulting plasma via an exemplary protocol as follows:
For a 25 μL reaction:
   0.625 μL Taqman Genotyping Assay (40× starting concentration, 1× final—contains custom primers/probes)
   3.75 μL BSA (10 mg/ml in 1× PBS, 0.05% Tween)
   8.125 μL Plasma containing cfDNA target
   12.5 μL Sensifast Probe Lo-ROX Mix (Bioline).
Thermal cycling protocol:
   Pre-read: 60° C., 30 seconds (1×)
   Initial denaturation: 95° C., 5 minutes (1×)
   35 cycles of: o Denaturation: 95° C., 10 seconds
      Anneal/Extend: 60° C., 50 seconds
      Post-read: 60° C., 30 seconds
   Hold: 4° C. (1×)

All reactions can be performed in triplicate on a Quant Studio 3 qPCR machine (Applied Biosystems).

Quantitatively circulating fragments spiked at 100% allelic frequency directly from plasma were detected (FIG. 24). In this example, the device was spiked, and the percent recovery at both 100% allelic frequencies, and lower allelic frequencies (5%, 1%, 0.1%) (representative of ctDNA allelic frequencies were measured.

### Example 3

### Nucleic Acid Purification using Magnetic Silica Beads

To ensure pure nucleic acid (NA) samples free of contaminants, many POC devices employ separate NA purification systems using the method of solid phase extraction based on silica membranes or particles. In this example, separate NA purification steps can be used in addition to the two-membrane filtering devices described herein.

Solid-phase extraction (SPE) is a sample preparation process by which compounds that are dissolved or suspended in a liquid mixture are separated from other compounds in the mixture according to their physical and chemical properties. In this case the liquid mixture is the sample and the compound to be isolated is the DNA. There are several SPE methods where the difference is embedded in the silica form that is used—microparticles, gel, or membrane. All of these techniques can be exploited both outside and inside the device. Here the general workflow of silica-based extraction method is presented, considering that all the methods are composed by the same main steps (binding, washing and elution).

The binding step can consist of mixing a binding buffer, usually a chaotropic agent, with the sample and the Silica. Chaotropic buffer is used since it's able to enhance the binding affinity between DNA and silica surface via hydrogen bonding and hydrophobic inter-actions. The chaotropic agent allows to make a phosphate group free from the DNA, and in the meanwhile the water present in the buffer protonates the Silica surface. These reactions make possible the binding between the silica and the DNA since the phosphate group becomes “exposed” and a hydrophobic interaction is possible.

The washing step allows for removal of other biological components bound to the silica surface and gets rid of the salts usually present in the binding buffer. Since the wash buffer is alcohol-based, the silica is left air dried for a while in order to make all the alcohol evaporate and not interfere with the next step.

The elution step re-hydrates nucleic acids so that they can be released from the membrane and are again free inside the solution. The elution buffer can often be just deionized water.

In this design, magnetic silica beads fixed with an external magnetic field to overcome issues with trapping small particles in very small (tens of microns) scale channels that often could overcome limitations due to deformation were used.

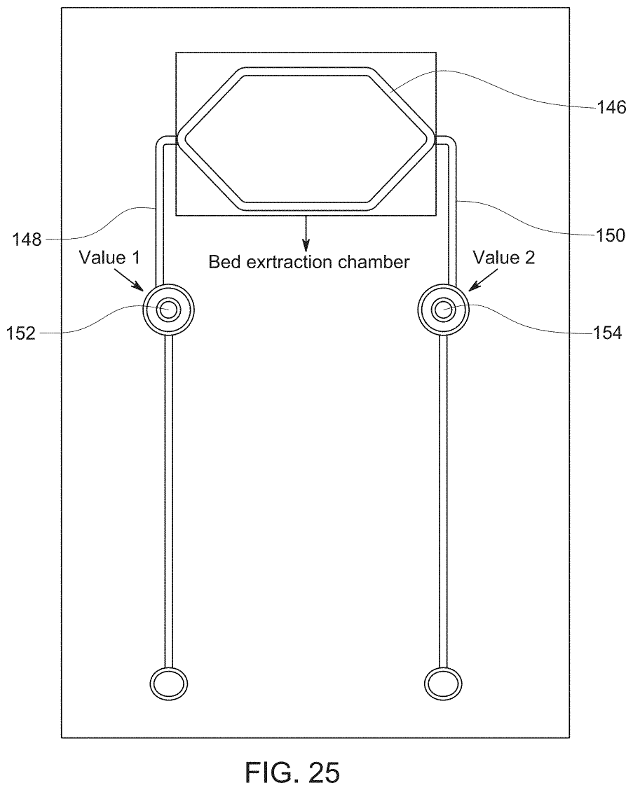

Fe₃O⁴ magnetic beads coated with silicon dioxide (2.5-4.5 μm) were purchased from G-Biosciences, and supplied in phosphate buffered saline, pH 7.4 with 0.09% Sodium Azide and 0.02% Tween-20. An exemplary NA purification device was designed as an exemplary hexagonal chamber 146 (11×5×1 mm) (also referred to herein as bed extraction chamber), connected to inlet 148 and outlet 150 through first valve 152 and second valve 154 (280×250 μm (width x depth) (FIG. 25) that allows better control of the flow.

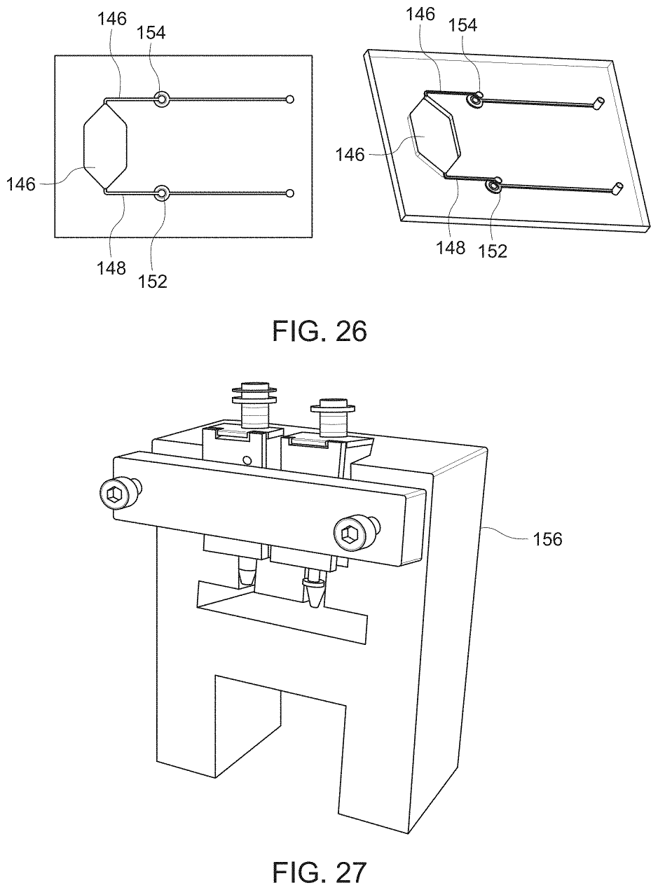

The exemplary device was designed using Solidworks® (see FIG. 26), and fabricated using a CNC micro-mill. A PMMA chip (25×34×1.5 mm) was used for fabrication, while the inlet and outlet were drilled manually and connected to external tubes to insert and remove fluids. Valves were designed using this configuration in order to have good control over the flow of different reagents. One side of the chip was bound to a PDMS layer, which can stop fluid flow when pushed down by a solenoid valve. The other side needed can also be bound in order to seal the channels on both sides and avoid leakages. Initially, a 0.2 mm PMMA layer provided substantially perfect sealing due to the high bonding force (PMMA-PMMA) was selected. Alternatively, an adhesive film was used because it also provided substantially good sealing (the back-pressure generated by the fluid motion was low enough to not break the bonding, even if the adhesion force was lower than the previous one), but the bonding procedure was much faster and cheaper. To facilitate bonding with the adhesive film, the device was washed with isopropyl alcohol (IPA) and bleach, dried with N₂, and then the layer was simply laid over the fabricated device, paying attention not to form air bubbles.

To exploit the valves function, a holder 156 for the solenoids (FIG. 27) was 3D printed and the device inserted before each extraction.

In one example shown in FIG. 28, the device was inserted in the solenoid holder 156, tubes connected to inlet 148 and outlet 150 and the magnetic stand placed under the chamber 146, then the following steps were done.
1. 43 μL of beads are inserted through the inlet, holding the valve 1 (V1) open and valve 2 (V2) closed (left panel).
2. The supernatant is sucked away by inspiration through a pipette by the outlet (both valves open).
3. 76 μL of dH2O flow through the valves (V1 and V2 open).
4. The remaining water is pumped out.
5. 40 μL of sample and 90 μL of Binding buffer are mixed in a tube and then 38 μL inserted.
6. The mixing is done by moving the magnet under the chamber and then the solution is left there for a couple of minutes (both valves close).
7. The fluid is pumped out.
8. Repeat 5-6-7 until the whole sample amount is consumed.
9. 38 μL of Wash buffer are inserted and pumped out.
10. Repeat step 9 twice.
11. 30 μL of pre-heated Elution buffer are loaded, mixing is done as before and kept there for 10 minutes.
12. The eluted sample is collected by sucking it out through a pipette.

In this example, the results are generated from the PCR product so to provide a comparison with the other methods. The mean value achieved was calculated and shown in Table 2. In this example, the volume of the fluid following nucleic acid purification can be adjusted to a desired volume. In addition, the volume of the fluid following nucleic acid purification can be adjusted to a desired volume (e.g., from an input volume of about 200 μL-1 mL to an output volume of about 5-50 μL).

A typical extraction curve is also reported in FIG. 29 with its related data in Table 1. The ratios point out that the sample is not completely pure, some contaminants are probably still present, but they are satisfying results since they are acceptable values.

TABLE 1
NA concentration extracted with microfluidic device (FIG. 29)

| NUCLEIC ACID | UNIT | 260/280 | 260/230 |
|---|---|---|---|
| 11.5 | ng/ml | 1.60 | 1.79 |

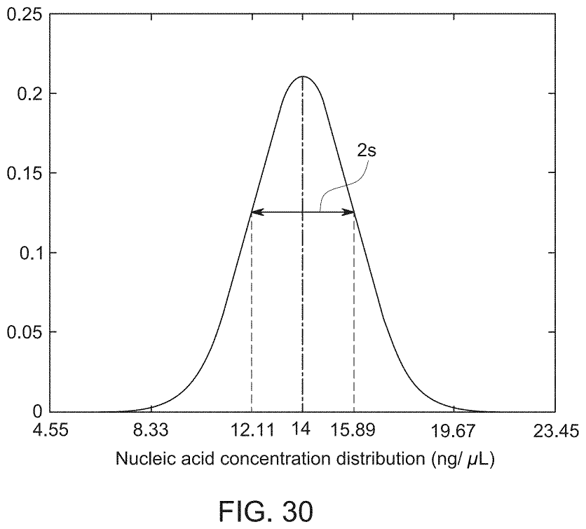

TABLE 2
Mean value, standard deviation and coefficient of variance
of NA concentration extracted on the device (FIG. 30)

| NUCLEIC | STANDARD | COEFFICIENT |
|---|---|---|
| ACID | DEVIATION | OF VARIANCE |
| (ng/ml) | (ng/ml) | (%) |
| 14 | 1.89 | 13.5 |

TABLE 3
Efficiency, standard deviation and coefficient of
variance achieved with DNA SPE on microfluidic device
(mean starting sample concentration 20 ng/ml)

| EFFICIENCY | Standard deviation | Coefficient of variance |
|---|---|---|
| (%) | (%) | (%) |
| 8.8 | 3.8 | 43 |

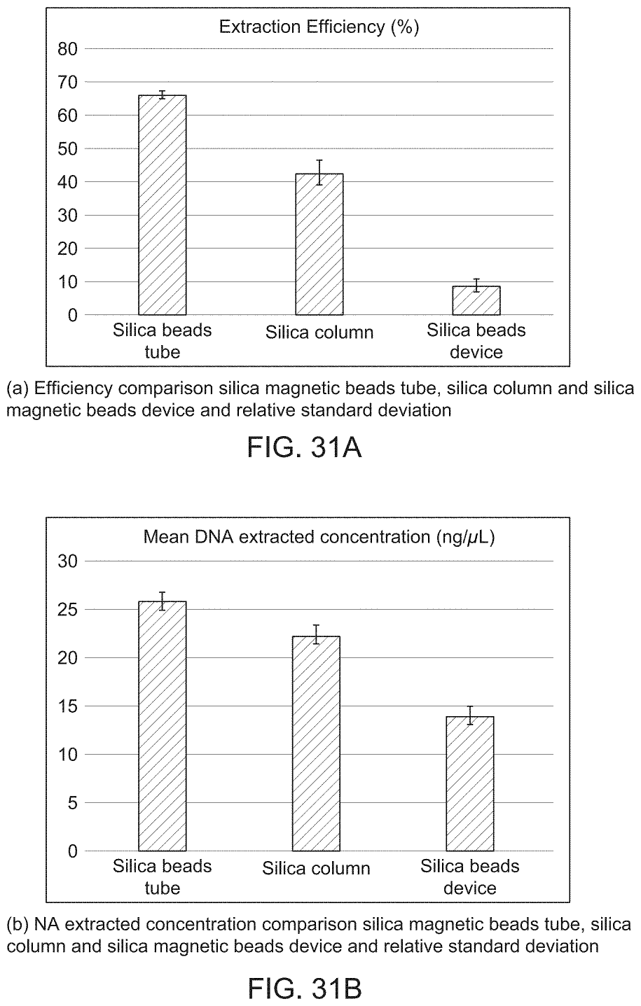

The summaries in FIGS. 31A and 31B show that the exemplary use of magnetic beads provide better results in the tube. However, moving the magnetic beads towards the device decreases performance. For example, it was difficult to collect the eluted sample directly from the outlet, and moreover if the outlet tube didn't fit exactly the hole, some leakage were observed. To address these problems, as discussed herein, a sample collection chamber for direct PCR could be used.

Without being bound by theory, it is believed that using just one inlet and one outlet, all the buffers and both the waste and eluted samples flowed in the same channels, which may have led to an unwanted mixing and the presence of impurities.

In another aspect, an exemplary microfluidic lab-on-a-chip device can consider the effective cfDNA concentration in a real sample is on the order of 100-0.1 ng/ml. However, this concentration was not detectable in this example by the ThermoScientific® Nanodrop 2000, which has a detection limit of 2 ng/μL for dsDNA.

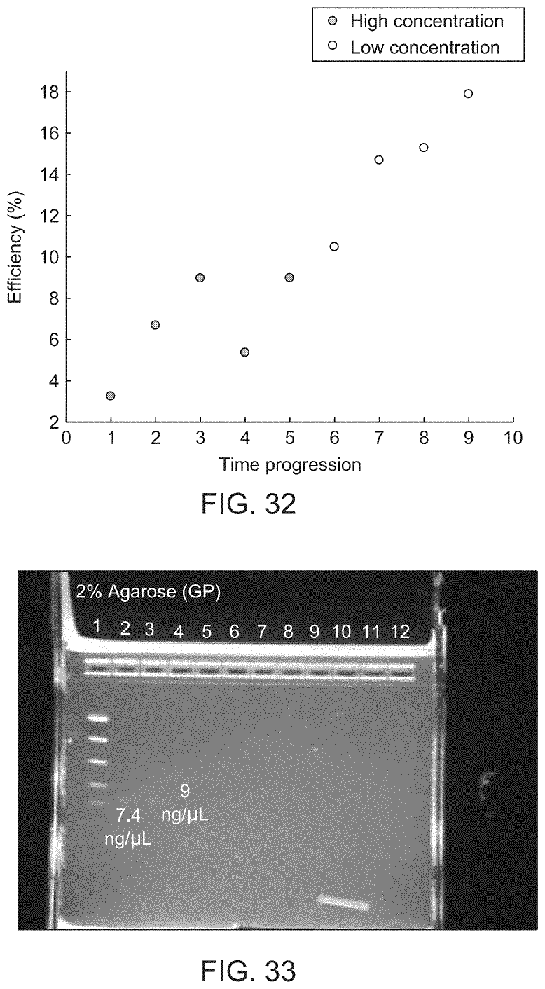

The following experiments were performed with a starting concentration on the order of 10 ng/μL, to identify a possible trend regarding performances change caused by decreasing the DNA concentration (FIG. 32).

TABLE 4
Efficiency, standard deviation and coefficient of
variance achieved with DNA SPE on microfluidic device
(mean starting sample concentration 10 ng/ml)

| EFFICIENCY | Standard deviation | Coefficient of variance |
|---|---|---|
| (%) | (%) | (%) |
| 11.9 | 4.9 | 41 |

The results (Table 4) are quite unexpected since they furnish higher efficiency than the one achieved with a higher starting sample concentration, but still with a very high standard deviation.

In FIG. 32 the efficiency trend is shown with respect to time progression, where the higher sample concentrations were employed before. As can be seen, the efficiency values increase over time.

In one aspect, a trade off could be found between the efficiency and the amount of sample used.

After the extraction procedure was optimized, PCR was performed on a purified sample, in order to understand if the two steps could actually be implemented one after the other in a final lab-on-chip (LOC) platform. If the extraction was successful, the same band found with the DNA template is found with the PCR product run over the agarose gel.

Two different samples are shown in FIG. 33, one with an initial concentration of 9 ng/μL and the other of 7.4 ng/μL. As can be seen both samples are correctly amplified, and no unexpected bands are present.

In another aspect, additional steps related to the extraction procedure and coupling the extraction procedure with the PCR over a unique device are provided.

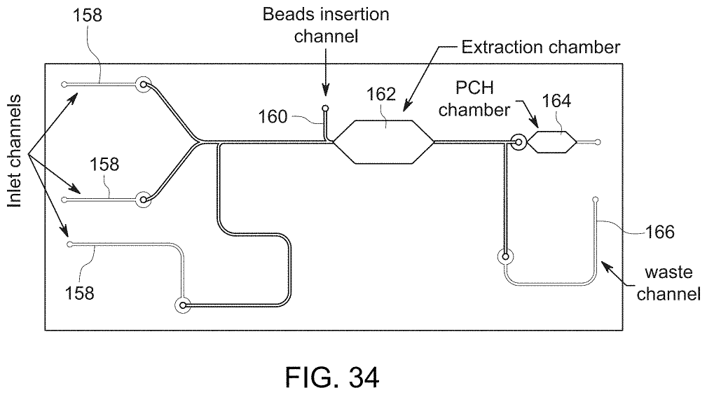

In this aspect, a new chip feature has been designed as an alternative to the above described devices. An exemplary device is shown in FIG. 34 where extraction chamber 162 is placed at the center of the chip with an adjacent short channel 160 for bead insertion and three inlets 158 for separate introduction of buffers to avoid unwanted mixing of reagents.

Optionally, PCR chamber 164 can be added to operate directly on the microfluidic platform after sample purification. Waste channel 166 can be added to discard waste. In another aspect, one of the inlets can be replaced with a channel (e.g., a long, serpentine channel) connecting two inlets to permit the mixing of the sample and the binding buffer directly over the chip (remember that in micro-scale the mixing of solutions is achieved just by diffusion, so a long channel is necessary, and a serpentine geometry enables to occupy less space as possible).

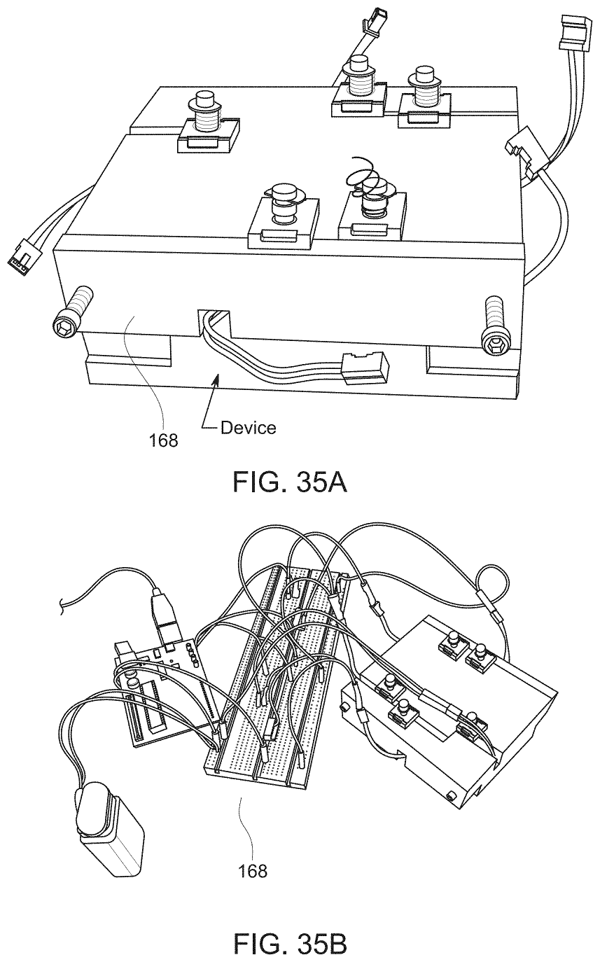

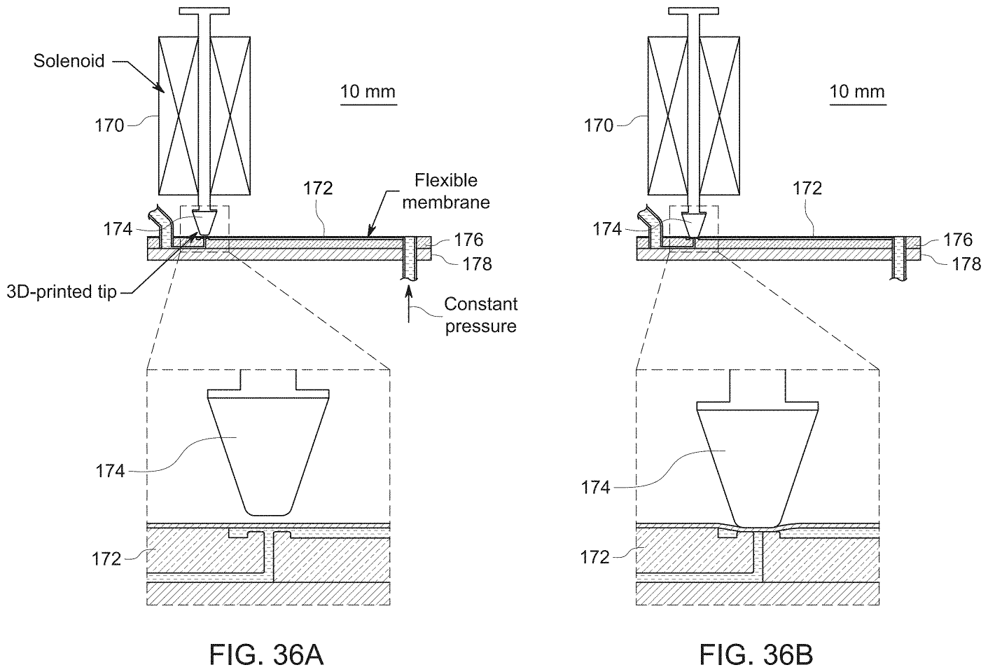

In another aspect, devices described herein can use valves to control flow of liquid through the system. Therefore, the correct functioning of the valves is very important, otherwise the solutions will be able to follow randomly the different channels. FIGS. 35A and 35B show an exemplary 3D device 168 as described above.

In one aspect, the sample is loaded and binding buffer (BB) (previously mixed) through one of the inlet leaving the related valve and the one of the waste channel open. In this way, the DNA will bind to the beads while the unwanted supernatant keep flowing and is discarded. The washing step works in the same way, but the inlet will be a different one. At the elution step the elution buffer (EB) is permitted to flow but the waste channel valve is kept closed, and the one of the PCR chamber open, so that the purified sample is collected.

### Example 4

### Solenoid Valve Design

The valve is one important part of microfluidic devices, especially for μTAS (micro total analysis system) “sample to answer” systems, and it is often compared to the transistor in the semiconductor industry. In one aspect, a solenoid valve compatible with a microfluidic PCR chip (sample to answer) that can withstand high pressures due to PCR is provided. This valve is compatible with a variety of materials (e.g., chips made entirely of plastic, a plastic-elastomer combination, or a plastic-adhesive combination).

In this aspect, a vertical valve actuated with a solenoid is provided. This valve design meets the requirements of a long-term stable, reliable, portable and low-cost valve for a fully-integrated point-of-care (POC) PCR device. The main drawback of solenoid actuated valves is the relatively large footprint required for each valve (roughly 1 cm²) and the lack of multiplexing capabilities (i.e. the ability to control multiple fluidic channels with a single valve). However, in this example, multiplexed valves were not used for two reasons: 1) multiplexing requires more complex designs with at least three layers, and 2) for this exemplary device, only 6-10 valves are required. An advantage of multiplexed valves is the ability to control 2n valves with 2n+2 actuators. Thus, for a chip containing 10 valves or less, it is unnecessary to increase design constraints to account for more complex multiplexed valves.

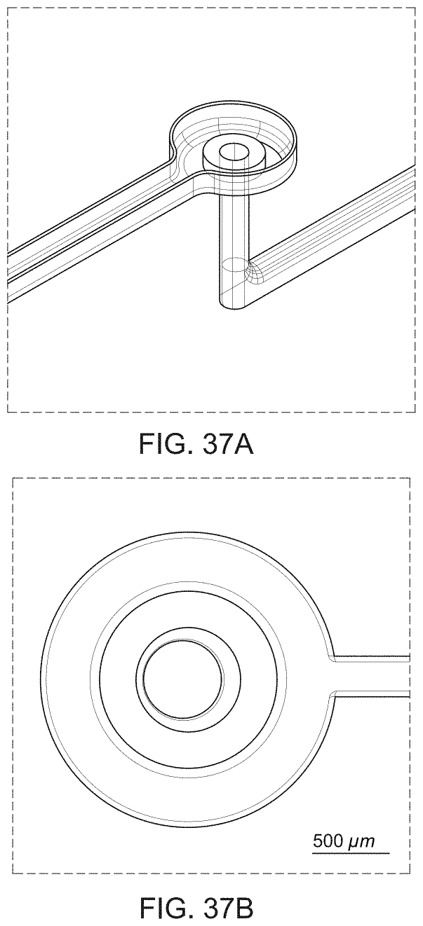

The valve van be manufactured by CNC micromilling, injection molding, and hot embossing. An exemplary working principle of the valve is explained in FIG. 36 having solenoid 170, flexible membrane 172, and tip 174. Flexible membrane 172 has a PMMA layer 176 and closing membrane 178. FIG. 36A shows a close up of tip 174 above flexible membrane 172 before application of pressure while FIG. 36B shows tip 174 in contact with flexible membrane 172 after contact. A 3D illustration of the valve is provided in FIG. 37A, and a photograph of the valve under the stereoscope can be seen in FIG. 37B. In this aspect, two different valves (PMMA-PDMS and PMMA-PMMA) were developed with this working principle.

PMMA-PDMS VALVE: This valve is made of two different polymers—an elastomer and a thermoplastic. One advantage of PDMS is its low Young's Modulus, which reduces the force needed to deform it and thus close the valve. In one aspect, the potential increased costs associated with PDMS production may be offset by the benefits of a thin membrane without features (e.g., channels, chambers, valves, etc.).

PMMA-PMMA VALVE: This valve is made of only one material, which is an advantage for the manufacturing and leads to fewer biocompatibility issues. However, the increased stiffness of the PMMA may require more force for deflection. An appropriate valve can be selected, for example, based on the desired biocompatibility, stiffness, and cost.

Based on models and given engineering restrictions in manufacturing, the following setups were tested:

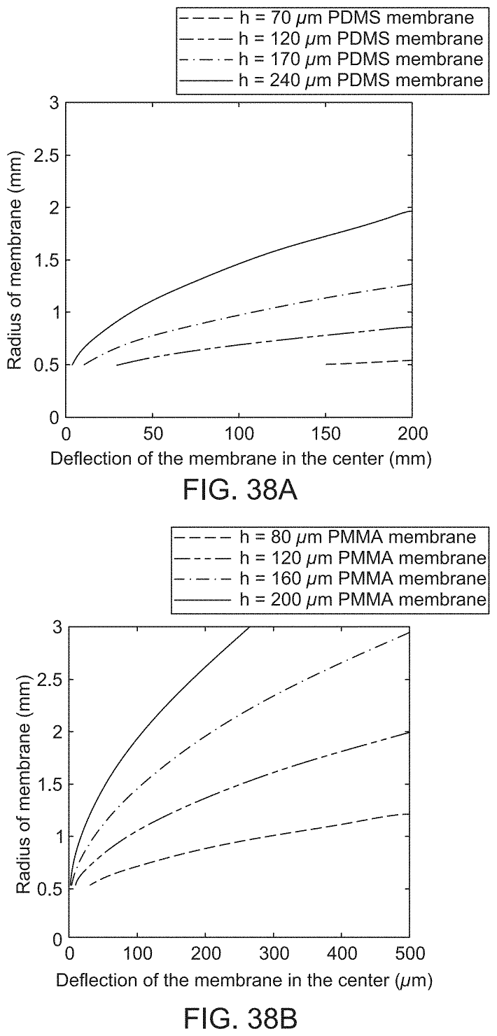

PMMA-PDMS (FIG. 38A)
Membrane radius of 1.1 mm; membrane thickness of 70 μm; distance between membrane and orifice of 200 μm
Membrane radius of 1.1 mm; membrane thickness of 140 μm; distance between membrane and orifice of 200 μm
Membrane radius of 1.1 mm; membrane thickness of 170 μm; distance between membrane and orifice of 200 μm
Membrane radius of 1.1 mm; membrane thickness of 240 μm; distance between membrane and orifice of 200 μm

PMMA-PMMA (FIG. 38B)
Membrane radius of 1.1 mm; membrane thickness of 80 μm; distance between membrane and orifice of 200 μm
Membrane radius of 2 mm; membrane thickness of 120 μm; distance between membrane and orifice of 200 μm

FIG. 38A shows the deflection of the PDMS membrane as a function of the radius for four different membrane thicknesses, and FIG. 38B shows the deflection of the PMMA membrane as a function of the radius for four different membrane thicknesses.

PMMA was chosen as material for the main body of the chip (as depicted in FIGS. 36A and 36B). The features on the top and on the bottom of all the chips were designed using SolidWorks CAD software and CNC micro-milled (HAAS Super Minimill). The microfluidic circuit was created using the Mastercam program. Milling conditions, (e.g., milling direction, spindle speed, feed rate, etc.) were optimized for surface quality. The fluidic ports were created using a drill press (Ellis) a radius of 300 μm. To connect the tubing to the chip, holes with a radius of 700 μm were drilled. The results presented below were generated with these chips.

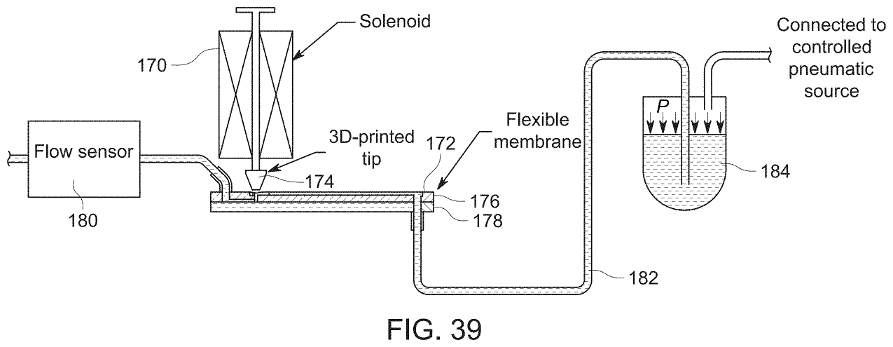

The setup is divided into an actuating and a sensing circuit. All devices were connected to an Arduino Uno and ultimately to a personal computer, in order to automate, synchronize and control the experiment and collect the data. FIG. 39 shows a scheme of the exemplary setup having flow sensor 180, solenoid 170, tip 174, flexible membrane 172, PMMA layer 176, closing membrane 178, outlet tube 182, and outlet collection chamber 184.

The push solenoid is from Adafruit Industries, LLC (Product ID: 2776). The pull-back spring of the solenoid was modified to have a spring constant of 0.024 N mm-1 in order for the plug to return to the off position when no voltage is applied.

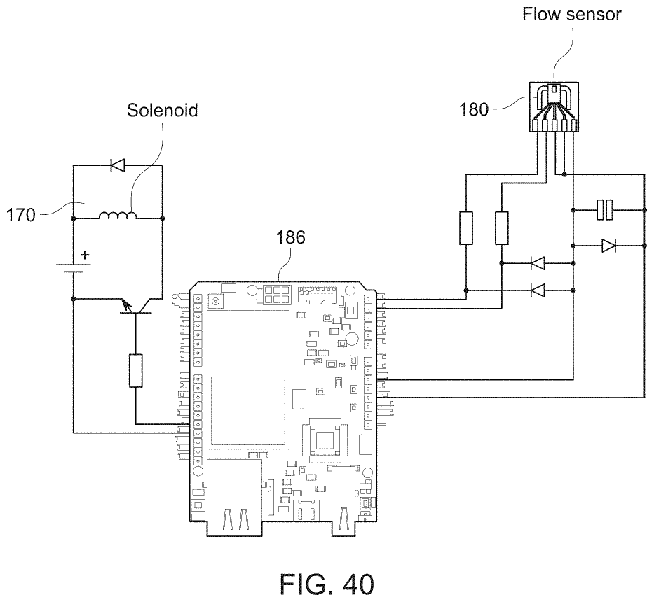

As shown in FIG. 40, Arduino Uno 186 would be sufficient to drive the solenoid. However, in order to make the valve portable and not depending on ground electricity it was actuated with a standard 9 V battery, which was controlled by the Arduino 186 using an NPN transistor (TIP102). In order to protect the circuit from a kick-back current a diode (1N4004) was used to connect the power channel with the solenoid ground leg. The solenoid was either actuated with a DC current and with Pulse Width Modulation (PWM), depending on the conducted experiment. To measure the flow rate across the valve a liquid flow sensor 180 (Sensirion LPG10) was connected to the outlet of the chip. Communication between the Arduino Uno and the flow sensor was set up taking advantage of the I2C interface. Programs written in C were adapted from and used to measure either at a high resolution (16 bit) with a delay between measurements of roughly 100 μs and a low resolution (9 bit) but a faster sample rate of roughly 10 μs. The Flow sensor detects flow rates below 150 μLmin-1 with a maximum error of ±0.188 μLmin-1. FIG. 40 shows a scheme of the sensing and actuating circuits.

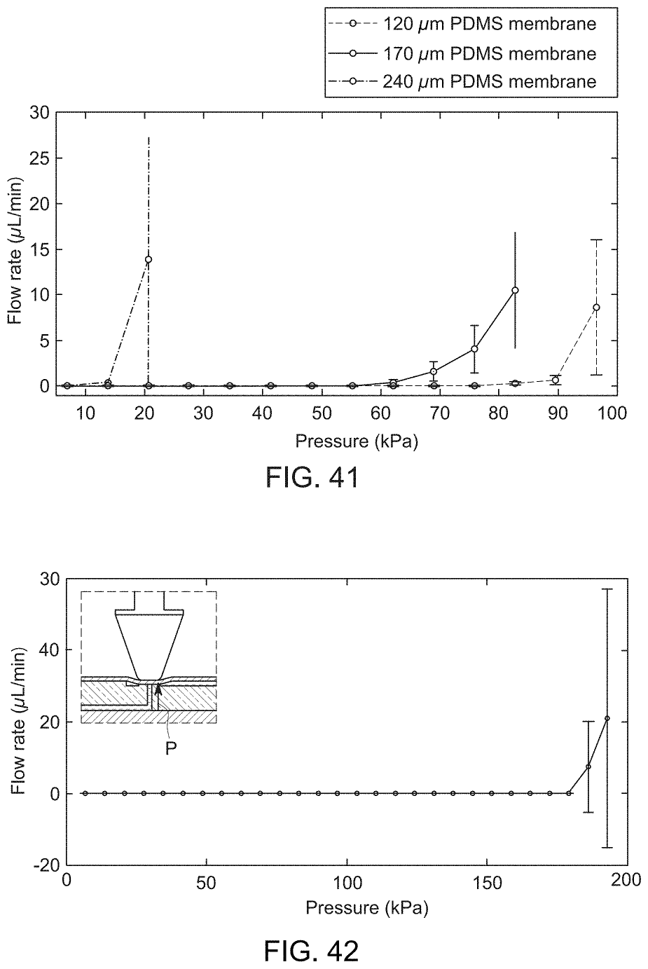

The thickness of the PDMS membrane was experimentally optimized evaluated by first applying a pressure in the horizontal directions at the inlet of the chip. For each PDMS membrane three identical experiments were conducted. The solenoid was actuated with 7.8 V DC during the entire experiment, and the leakage across the valve was measured with the flow sensor. Pressure in the range from 0-100 kPa in increasing in steps of 6.8 kPa was applied for 10 s per step, and PDMS membranes with thicknesses of 70, 120, 170 and 240 μm were tested. For each pressure step 120 data points were collected but only 60 were taken into consideration, since the first and the last 2.5 s of each pressure step were neglected to consider the time slot that was taken for precise manual pressure adjustment. From a total of 180 (60 times three experiments) data points the average and the standard deviation were calculated and plotted in FIG. 41. The results for the 70 μm membrane were not reported as, in this aspect, the membrane burst at very low pressures.

Furthermore, the PMMA-PDMS valve was tested with a pressure applied at the outlet of the chip, in vertical direction, as indicated in the upper left corner in FIG. 42. This is, for example, of relevance for a PCR, which needs to be sealed against a pressure build up during the temperature cycling. The experiment was conducted under the same conditions as mentioned above, but in a pressure range from 0-200 kPa an only the 120 μm membrane was examined. The results are depicted in FIG. 42.

FIG. 41 shows that the valve with the 120 μm membrane delivers the best results, which is in accordance with theoretical equations. However, the less ideal performance of the 240 μm membrane was not expected. The valve is more stable in vertical direction, because the area to which the acting pressure of the liquid is applied is smaller, resulting in a lower force. The relatively large standard deviations above the valves working range are due to the fact, that three experiments were conducted and in every experiment the valve behaves slightly different.

This can be a result of variations in the membrane thickness, fluctuations in the battery power and pressure adjustment errors.

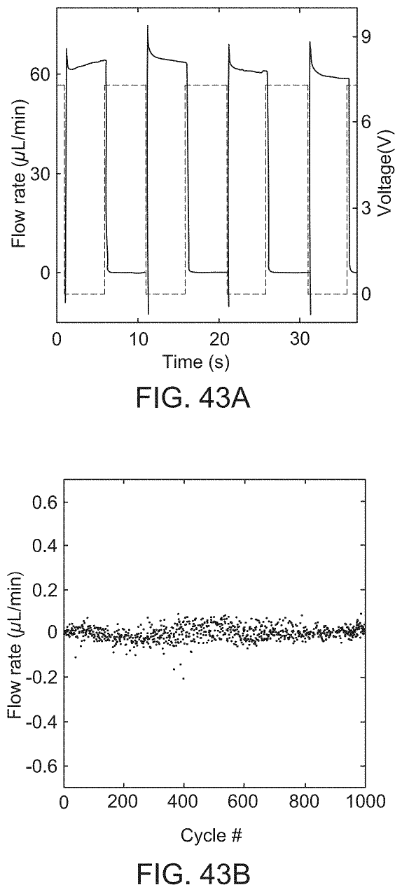

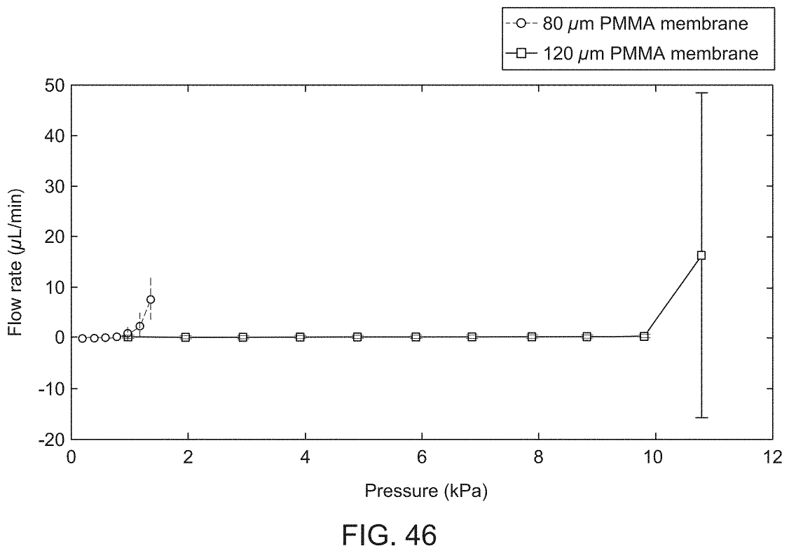

Reliability of Valves: To demonstrate the working principle, valve on/off cycles of 5 s each were run. The data in FIG. 43A is from one experiment with 12 data points per second leading to 120 data points per on/off interval. A pressure of 2.5 kPa was applied, the solenoid was actuated with 7.8 V DC and a 120 μm membrane was tested. The plot shows sharp transitions and demonstrates that the valve closes upon application of a voltage, and vice versa. To evaluate the reliability and long-term stability of the valve, 1000 on/off (2 s/8 s) cycles were run over 167 min (FIG. 43B). Each data point in the plot represents the average of 12 collected measurements in the on state. The results are plotted in FIG. 46 on the right and show that the valve is highly reliable. Out of 1000 cycles, no measurement was above or below 0.2 μL/min and −0.2 μL/min, respectively. Error bars are not shown because they were too small to be visible.

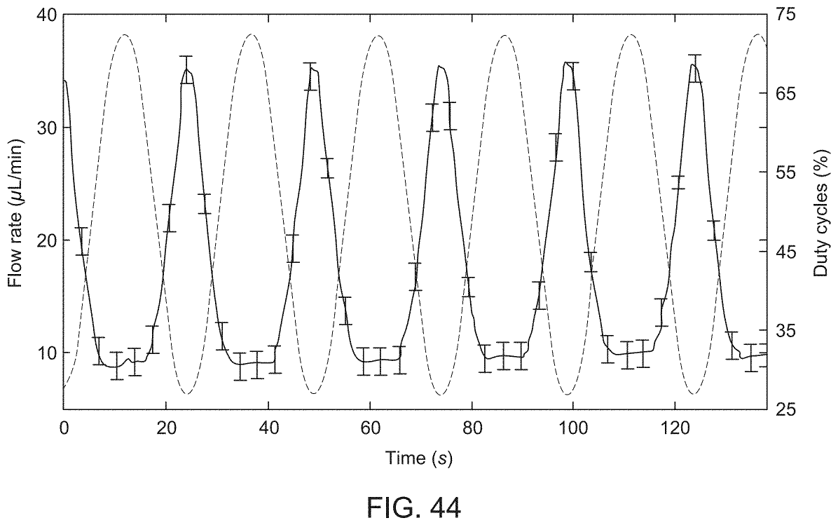

Duty Cycles and Response Time: Typically, solenoids are used as digital (on/off) actuators. If a DC voltage is applied the plunger of the solenoid moves out at maxi-mum speed until it come in contact with the membrane. This leads to two undesired effects. 1) the valve wears out and 2) The impulse is transferred to the fluid in the channel. Thus, a certain control over the force of the plunger is desirable. This is achieved by applying a PWM signals with different duty cycles. For the experiment depicted in FIG. 44 a sinusoidal signal of duty cycles in the range of 27-73% was applied at a frequency of 976.56 Hz with a maximum voltage of 7.8 V. The pressure acting on the fluid was 3.5 kPa and the PDMS membrane 120 μm in thickness. The flow rate replicates the input signal, this experiment thus demonstrates that control over the force of the plunger can be achieved.

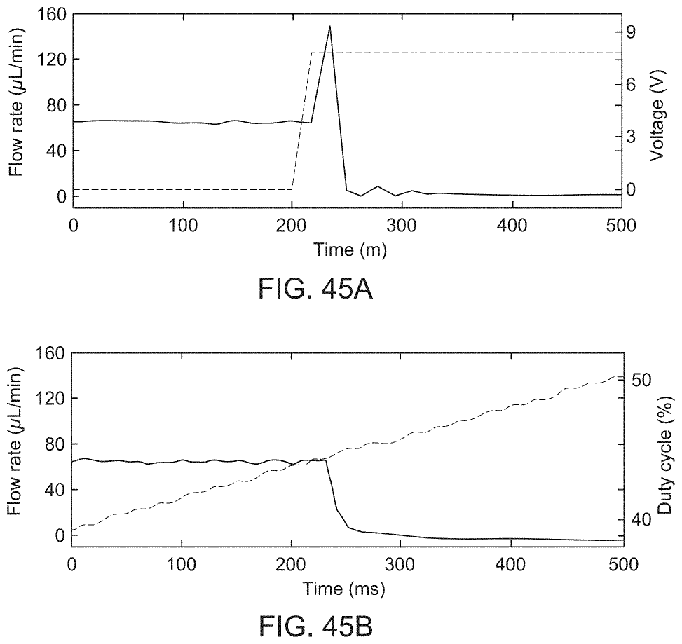

FIGS. 45A and 45B show the flow rate across the valve as response to a DC and a PWM voltage applied to the valve. The former shows a rapid increase in the flow rate after the plunger contacted the membrane due to the impulse transfer from the solenoid to the liquid. The latter, however, shows a much smoother transition with duty cycles increasing from 40 to 50%. For either actuation method, the response time is below 100 ms.

For the PMMA-PMMA valve, the model showed that an 80 μm PMMA membrane with a radius of 1.1 mm and a 120 μm PMMA membrane with a radius of 2 mm can sufficiently be deflected in order for the valve to shut the liquid flow and with-stand the pressure. To experimentally examine the pressure resistance, three identical experiments for each of the two PMMA valves were performed. The solenoid was actuated with 7.8 V DC. The applied pressure ranged from 0-11 kPa with steps of 0.2 and 1 kPa, respectively. For every pressure step, 120 data points were collected and 60 were taken into consideration. The first and the last 2.5 s of each pressure step were neglected considering the time slot required for precise manual pressure adjustment. From the total of 180 data points the average and the standard deviation were calculated and plotted in FIG. 46. FIG. 46 shows that the valve with the 1.1 mm PMMA membrane is stable up to 0.8 kPa. The valve with the 2 mm PMMA membrane is stable for up to 10 kPa. Two experiments demonstrated valve stability for pressures up to 18 kPa. In one experiment, however, the valve started leaking at around 11 kPa.

Power: The voltage (V), current (I) and force at full stroke (Fmax) of the solenoid were measured for duty cycles from 0-100% at 976.56 Hz with a multimeter and a scale, respectively. The operating time was then calculated with a standard value for the charge capacity C=620 mAh of a 9 V battery. The results are shown in Table 5.

TABLE 5
The power consumption, operating time and force at full
stroke for a solenoid as a function of the duty cycle
for a 9 V Battery with a capacity C of 620 mAh.

| Duty Cycle | Power | Operating Time | Maximum Force |
|---|---|---|---|
| (%) | (W) | (h) | (N) |
| 0 | 0.00 | ∞ | 0.00 |
| 10 | 0.20 | 1.26 | 0.10 |
| 20 | 0.82 | 0.83 | 0.60 |
| 30 | 1.70 | 0.70 | 1.60 |
| 40 | 2.86 | 0.62 | 2.30 |
| 50 | 4.12 | 0.55 | 2.80 |
| 60 | 5.71 | 0.50 | 3.40 |
| 70 | 7.50 | 0.46 | 3.70 |
| 80 | 9.49 | 0.43 | 4.00 |
| 90 | 10.88 | 0.41 | 4.50 |
| 100 | 11.70 | 0.41 | 4.90 |

Overall, the size of the valve was restricted by a hole that had to be drilled in vertical direction to connect the channels on both sides of the PMMA chip. Thus, in this example, the minimal radius of the valve was 1.1 mm. The thickness of a valve made of PMMA or PDMS can be optimized. In this aspect, the best results were achieved with a PDMS membrane of 120 μm in thickness. This valve was stable for up to 75 kPa in the horizontal direction and up to 175 kPa in vertical direction. Furthermore, it worked reliably for more than 1000 actuation cycles. The Young's modulus of PMMA is more than 1000-fold larger than the one of PDMS. Consequently, the valve was not as pressure resistant as the valve with the PDMS membrane, however, still worked for smaller pressures. The valve with the smaller radius and the 80 μm membrane withstood pressure up to 0.8 kPa and the valve with the larger radius and the 120 μm membrane up to 10 kPa. While the smaller valve did not show a stable behavior over multiple cycles, the larger valve proved to be reliable for more than 100 actuation cycles.

The exemplary valves described herein provide both portability and their reliability. The PMMA-PMMA version is inexpensive for large scale production what makes it an attractive valve for a commercial device. The PMMA-PMDS valve is still manufacturable at an attractive price and applicable to high-pressure ranges. The valves are furthermore bio- (and, specifically, PCR) compatible and have a short response time. The valve can be applied on both sides of the chip, which, theoretically, doubles the number of valves per chip.

TABLE 6
Comparison of the reviewed valves and the valves described herein.

| | | Response | | Back- | | | | |
|---|---|---|---|---|---|---|---|---|
| Valve Type | Source | Time | Biocompatibility | pressure | | Automatability | Scalability | Portability |
| MEMS-valves | [20] | ✓ | X | — | | ✓ | ✓ | ✓ |
| Pneumatic valve | [22] | ✓ | ✓ | 70 | kPa | ✓ | ~ | X |
| TWIST-valves | [25] | X | ✓ | 500 | kPa | X | ~ | ✓ |
| Hydraulic valves | [26, 27] | ✓ | ✓ | — | | ✓ | ~ | ✓ |
| Magnetic valves | [28] | ✓ | ~ | 1.5 | kPa | ✓ | ~ | ✓ |
| 3D printed valves | [30] | ✓ | ✓ | — | | ✓ | X | X |
| Smart material | [31] | X | ✓ | 2 | kPa | ✓ | ~ | X |
| valves | | | | | | | | |
| PMMA-PDMS | — | ✓ | ✓ | 75 | kPa | ✓ | ~ | ✓ |
| valve | | | | | | | | |
| PMMA 1.1 mm | — | ✓ | ✓ | 0.8 | kPa | ✓ | ✓ | ✓ |
| valve | | | | | | | | |
| PMMA 2 mm | — | ✓ | ✓ | 10 | kPa | ✓ | ✓ | ✓ |
| vaive | | | | | | | | |

### References for Above Table:

[20] Kwang W Oh and Chong H Ahn. “A review of microvalves”. In: Journal of micromechanics and microengineering 16.5 (2006), R13.
[22] M A Unger et al. “Monolithic microfabricated valves and pumps by multilayer soft lithography.” In: Science 288.5463 (2000), pp. 113-116.
[23] Pan Gu, Toshikazu Nishida, and Z. Hugh Fan. “The use of polyurethane as an elastomer in thermoplastic microfluidic devices and the study of its creep properties”. In: Electrophoresis 35.2-3 (2014), pp. 289-297.
[25] Douglas B. Weibel et al. “Torque-actuated valves for microfluidics”. In: Analytical Chemistry 77.15 (2005), pp. 4726-4733.
[26] Yi Chung Tung and Shuichi Takayama. “Ionic liquids for microfluidic actuation: Multiplexed hydraulic valve actuation using ionic liquid filled soft channels and braille displays”. In: ACS Symposium Series 1030 (2007), pp. 157-173.
[27] Kweku a Addae-Mensah et al. “Actuation of elastomeric microvalves in point-of-care settings using handheld, battery-powered instrumentation.” In: Lab on a chip 10 (2010), pp. 1618-1622.
[28] William C Jackson et al. “Rapid prototyping of active microfluidic components based on magnetically modified elastomeric materials”. In: Journal of Vacuum Science & Technology B: Microelectronics and Nanometer Structures Processing, Measurement, and Phenomena 19.2 (2001), pp. 596-599.
[30] Anthony K Au et al. “3D-printed microfluidic automation.” In: Lab on a chip 15.8 (2015), pp. 1934-41.
[31] Lin Gui et al. “Microfluidic phase change valve with a two-level cooling/heating system”. In: Microfluidics and Nanofluidics 10.2 (2011), pp. 435-445.

### Example 4

### Stronger Valves

The valves discussed above form a strong foundational basis for similar valves with different types of solenoids (off shelf). In another aspect, a continuous push solenoid with a 0.25″ stroke distance and 1 oz force (McMaster-Carr, Product #699905K172) was used. This solenoid can provide different forces at different percentages of the stroke length and can be actuated by 12 V DC. A circuit diagram for a single solenoid valve is shown below.

The use of these adjusted solenoid valves reduces overheating of the valves and is able to withstand full pressure build-up of PCR in a microfluidic chip. Further, valves with PDMS and adhesive sealings were successfully tested.

### Example 5

### PCR Chip

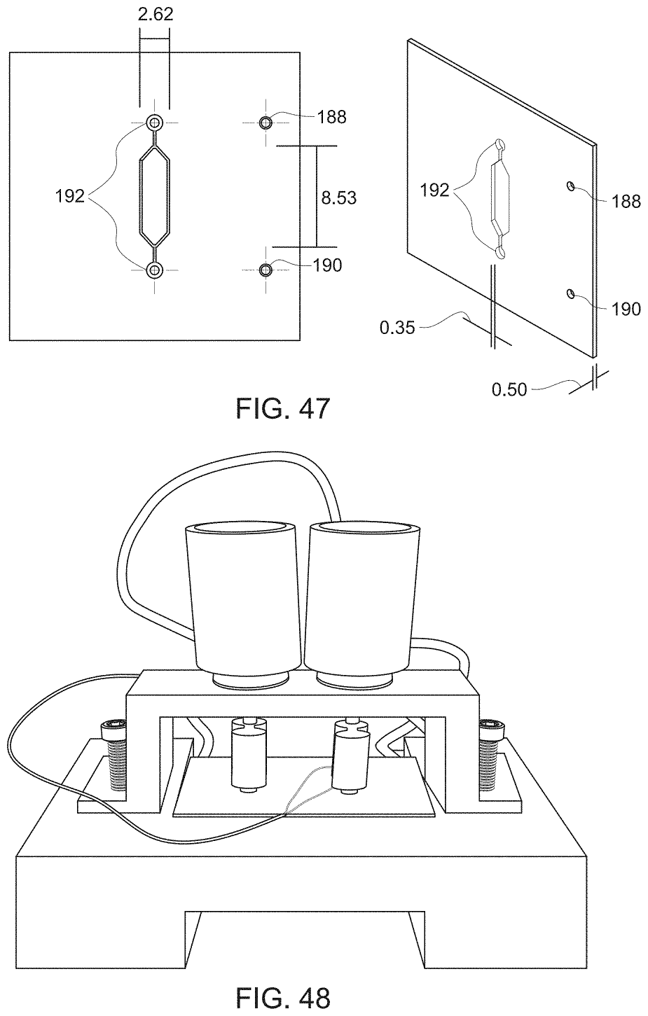

Design of chip: These valves are compatible with an exemplary plastic, microfluidic chip capable of conducting PCR as described herein. These chips are made using PMMA and have features on both sides of a single layer, making them small, easy to manufacture, and easy to use once made. As shown in FIG. 47, the exemplary PCR chip has inlet 188, outlet 190 for receiving fluid and conducting fluid out of the PCR chip, respectively. The PCR chip also has valves 192 near hexagonal PCR chamber 194. The PCR chip can be sealed with plastic, elastomer, or a simple adhesive.

These valves have been designed to be compatible with vertical solenoid valves described above. An image of the chips, valves, and 3D printed holder is shown below:

As shown in FIG. 48, the exemplary valves and chips can also be fit with custom made adapters branching into three parts to actuate three valves at once.

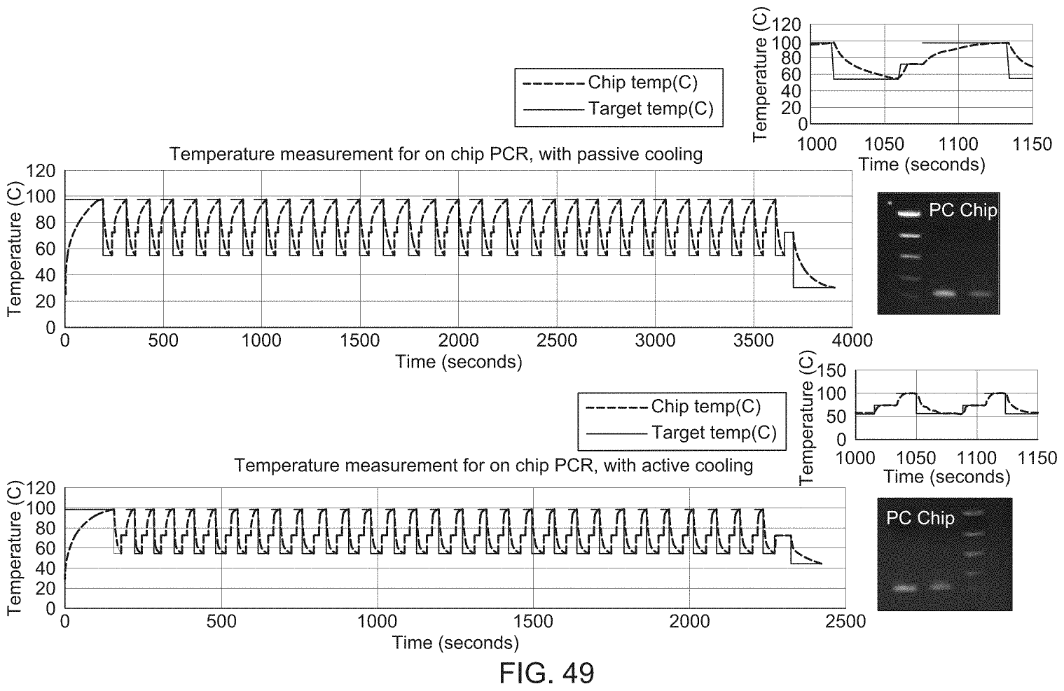

In terms of electronic components necessary to conduct PCR, a small Peltier heater and K-type thermocouple for heating, cooling, and temperature sensing can be used. These components were selected because they are cheap, accurate in small temperature ranges, and can be reused without touching. In one aspect, the chip is designed to be fully sealed—the thermocouple is able to provide a very accurate approximation of the temperature inside the PCR chamber even when it is not in contact with the fluid, which is possible due to the optimized design of the chip. PWM and active cooling can be integrated using this very low-cost, minimal equipment set-up. A thermocouple was sandwiched between the heater and the chamber of the chip to measure the temperature and plotted as compared to the target temperature (FIG. 49). FIG. 49 also shows the PCR products on an electrophoresis gel to confirm the presence of expected PCR products.

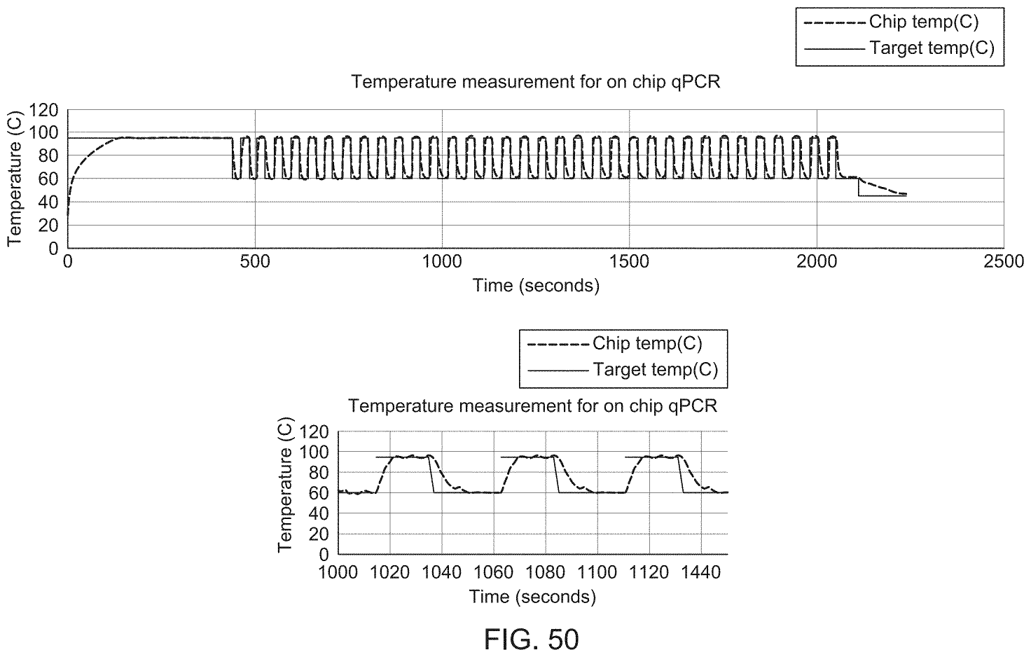

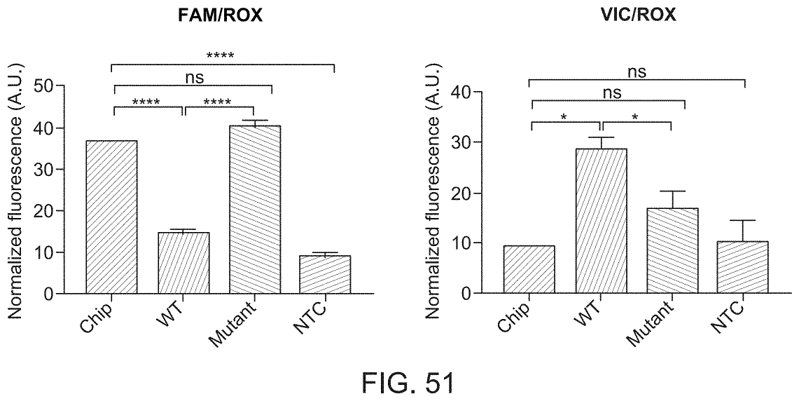

A custom Arduino program was written for this example, however, it is very simple to re-write the program for other thermal conditions, such as isothermal amplification, which may be useful for additional on-chip experiments. For instance, qPCR protocol was performed on this chip and successful results were obtained that are comparable to the gold-standard device (Quant Studio 3, Applied Biosystems). FIG. 50 shows thermal cycling results with a 2-step protocol with active cooling on chip. FIG. 51 shows endpoint fluorescence results from an on-chip PCR product compared to the gold standard qPCR product. All PCR products analyzed in a plate reader to measure endpoint fluorescence. These results show single nucleotide changes can be distinguished using the exemplary on-chip PCR (FIGS. 50 & 51).

Further, proof-of-concept compatibility between valving components and PCR has been demonstrated. However, these valves and chips can be actuated in pulse patterns such that fluid flow can be controlled for use when integrating additional functionalities to the device, such as sample preparation and signal detection.

While aspects have been disclosed with reference to certain embodiments, numerous modifications, alterations, and changes to the described embodiments are possible without departing from the sphere and scope of the present disclosure, as defined in the appended claims. Accordingly, it is intended that the present disclosure not be limited to the described embodiments, but that it has the full scope defined by the language of the following claims, and equivalents thereof.

What is claimed is:

1. A blood sample preparation system comprising:
a filter component comprising at least a first filter and a second filter for preparing a blood sample, each filter having a different pore size; and
a microfluidic amplification component for amplification of nucleic acid from the blood sample.

2. The blood sample preparation system of claim 1, wherein a volume of the blood sample is greater than about 100 μl.

3. The blood sample preparation system of claim 1, wherein the microfluidic amplification component is in fluid connection with the filter component.

4. The blood sample preparation system of claim 1, further comprising a DNA purification component.

5. The blood sample preparation system of claim 1, wherein the filter component further comprises:
an inlet component having an inlet port for receiving a blood sample;
an outlet component having an outlet port for releasing a filtered blood sample; and
a filter support for retaining a first filter and a second filter.

6. The blood sample preparation system of claim 5, wherein the first filter and second filter have a pore size, and the pore size of the first filter is larger than the pore size of the second filter.

7. The blood sample preparation system of claim 6, wherein the pore size of the first filter and the second filter range from about 1 μm to about 25 μm.

8. The blood sample preparation system of claim 1, wherein the microfluidic amplification component comprises a polymerase chain reaction chamber for amplifying nucleic acid.

9. The blood sample preparation system of claim 8, wherein the microfluidic amplification component further comprises at least two inlets for the polymerase chain reaction chamber for providing reagents to the polymerase chain reaction chamber.

10. The blood sample preparation system of claim 9, wherein the microfluidic amplification component further comprises an extraction chamber disposed between the at least two inlets and the polymerase chain reaction chamber for extracting nucleic acid from the blood sample.

11. The blood sample preparation system of claim 8, wherein the microfluidic amplification component further comprises an outlet from the polymerase chain reaction chamber for disposal of waste products.

12. The blood sample preparation system of claim 8, wherein the microfluidic amplification component further comprises at least one valve for controlling flow of reagents to and from the polymerase chain reaction chamber.

13. The blood sample preparation system of claim 12, wherein the at least one valve is a solenoid valve.

14. The blood sample preparation system of claim 13, wherein the solenoid valve comprises a sensing tip.

15. The blood sample preparation system of claim 8, wherein the microfluidic amplification component further comprises an actuator.

16. The blood sample preparation system of claim 15, wherein the actuator comprises a membrane.

17. A method of filtering and amplifying a nucleic acid target from a blood sample, comprising:
providing a blood sample to a filtering component;
filtering the blood sample through a first filter and a second filter to form a filtered blood sample; and
amplifying the nucleic acid target from the filtered blood sample.

18. The method of claim 17, wherein the first filter and the second filter have a pore size, and the pore size of the first filter is larger than the pore size of the second filter.

19. The method of claim 18, wherein the pore size of the first filter and the second filter range from about 1 μm to about 25 μm.

20. A method of filtering a blood sample and amplifying a nucleic acid target from the blood sample, comprising:
providing a blood sample to a filtering component;
filtering the blood sample through a first filter and a second filter to form a filtered blood sample;
purifying nucleic acid from the filtered blood sample to form a purified nucleic acid sample; and
amplifying the nucleic acid target from the purified nucleic sample.
